SEQUENCE LISTING (2 sequences)

SEQ ID NO: 1 - protein, 315 aa, synthetic construct
MALDPHLAGVLQQLAAANRKSTAEGTPEEGRAGYLALTRGSLTPEQIVPVASVQDTTVPGGAGPVAARIYRPEGAGPFPTVAYFHGGGYVIGNLDTHDNICREICRGAQAVVVSVDYRLAPEHPFPAGIEDAVAAARWVVANAHALGGSATVAVAGDSAGGNFCAVVTQQLRDAGIALAAQFLIYPAVDHAAAEYASAEQNAKGYFLEAETMAWFYNHYAGTFPDALDPRLAPLQAKSLANLPSAVIVNAEFDPLRDQGAAYAEALRAAGGQAELIEGAGMIHGFFDMGRWSPGAQAVITHSIERFAALLTTRAR

SEQ ID NO: 2 - DNA, 4408 bp, synthetic construct
  source 1..4408: mol_type = other DNA
ccaatgatactgatttttaaggcgactgatgagtcgccttttttttgtctaagaattcatcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatccgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttggagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccgggacgtcgtgccccaactggggtaacctttgagttctctcagttgggggatcgatagtcaaaagcctccggtcggaggcttttgactagcacctcggtaccaaattccagaaaagaggcctcccgaaaggggggccttttttcgttttggtccggatccgatatcagtctctatggaggtcaggtatgattactattgacaattaatcatcggctcgtataatgtgatcagacctggaattgtgagcggataacaattgttctacgggtaagggggtttttttatggcgctggatccacatctagccggcgttctccagcaacttgcggctgcgaaccgcaagtcaaccgctgaggggacccctgaggaaggccgagcggggtatctcgccttaacgagagggagcttaacgccagaacagatcgttccagtcgcgagtgtacaggatacaaccgtaccgggtggagccggccctgtggcagcgcgcatctatagacctgaaggggcgggaccgtttcctacagtggcgtacttccatggcggtgggtacgtcatcggtaatttggatacacacgacaatatctgccgtgaaatttgtcgcggtgcccaagctgttgtggttagtgtggactatcggttagccccggaacatcccttcccagccggaattgaagatgcagtagcggcagctcggtgggtcgtcgcgaacgctcacgcgttgggcggatcagccactgtagcagtggcaggggacagcgcaggcgggaatttctgtgcggtggtaacccagcagttgcgcgacgcaggcatagctctagctgcgcagttcttgatttaccccgcggttgaccacgcggcggcggagtacgcttcagcagaacaaaatgccaaaggctattttctggaggcagaaacgatggcttggttctacaaccattacgcaggcaccttccctgacgcccttgatccccgcttggctcccttacaagcaaagagtctggctaacttgccgtccgctgtgattgttaacgccgaattcgacccactaagagatcaaggtgccgcttacgccgaggcattaagagccgctggtgggcaagcagagttgatcgagggcgctggcatgatccacgggttctttgatatggggagatggtcgccgggagcgcaggcggttataacacatagtatagaacgttttgccgccctgttgacaactagggctcgataatctagactcgaggacgaacaataaggcctccctaacggggggccttttttattgataacaaaaatccacaaggaaaaattaaaggggagataaaatcccccctttttggttaactgcggccgcgtcgtggtttgtctggtcaaccaccgcggtctcagtggtgtacggtacaaaccccgacgctagctgcgggtgccagggcgtgcccttgggctccccgggcgcgtactccatcggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

(19) United States
(12) Patent Application Publication
(10) Pub. No.: US 2026/0078387 A1
(43) Pub. Date: Mar. 19, 2026

(54) ENGINEERED BACTERIA, SYSTEMS, AND METHODS FOR DEGRADING POLYESTER MATERIALS

(71) Applicants: Northwestern University, Evanston, IL (US); The University of Chicago, Chicago, IL (US); UT-Battelle, LLC, Oak Ridge, TN (US)

(72) Inventors: Ludmilla E. Aristilde, Evanston, IL (US); Rebecca Ann Wilkes, Evanston, IL (US); Jacob R. Waldbauer, Chicago, IL (US); Austin L. Carroll, Oak Ridge, TN (US); Adam M. Guss, Oak Ridge, TN (US); Nanqing Zhou, Evanston, IL (US)

(21) Appl. No.: 19/333,241

(22) Filed: Sep. 18, 2025

Related U.S. Application Data
(60) Provisional application No. 63/695,951, filed on Sep. 18, 2024.

(51) Int. Cl. C12N 15/78; C08J 11/18; C12N 1/20; C12N 9/18; C12R 1/40

(52) U.S. Cl. CPC C12N 15/78; C08J 11/18; C12N 1/20; C12N 9/18; C12R 2001/40

(57) ABSTRACT

Disclosed are engineered bacteria, systems, and methods for degrading polyester materials, including materials having polyethylene terephthalate (PET).

### CROSS-REFERENCE TO RELATED APPLICATION

This application claims the benefit of U.S. Provisional Application Ser. No. 63/695,951, filed Sep. 18, 2024. The content of which is incorporated by reference in its entirety.

### STATEMENT REGARDING FEDERALLY SPONSORED RESEARCH OR DEVELOPMENT

This invention was made with government support under grant number CHE-2109097 awarded by the U.S. National Science Foundation and grant number DE-AC05-00OR22725 awarded by the U.S. Department of Energy. The government has certain rights in the invention.

### REFERENCE TO SEQUENCE LISTING

The contents of the electronic sequence listing (70258102715.xml; Size: 7,383 bytes; and Date of Creation: Sep. 12, 2025) is herein incorporated by reference in its entirety.

### BACKGROUND

Ubiquitous and extensive usage of plastic materials has led to the accumulation of plastic wastes, which are projected to reach ˜33 billion tons by the year 2050. Plastic wastes have been reported in various environments including marine and fresh waters, sediments, and soils. Microplastics (MPs) and nanoplastics (NPs), defined operationally as plastic fragments with sizes smaller than 5 mm and 1 μm, respectively, are considered a threat to both aquatic and terrestrial ecosystems, as organisms can easily ingest these small particles. Since plastic wastes are entirely anthropogenic, wastewater treatment plants (WWTP) represent important repositories for plastics, contributing to their release into natural systems and serving as a source of MPs and NPs.

### SUMMARY

In one aspect, an engineered bacterial strain is provided. The engineered bacterial strain can include one or more heterologous hydrolase capable of degradation of at least a portion of a polyester material.

In another aspect, a method of fragmentation and/or degradation of at least a portion of a polyester material is provided. The method can include exposing the polyester material to one or more hydrolase from Comamonas testosteroni.

### BRIEF DESCRIPTION OF THE FIGURES

FIGS. 1A-1E. Extent of biofragmentation of PET films and pellets by C. testosteroni KF-1. (FIG. 1A) Schematic overview of the investigated steps in PET plastics biodeterioration, biofragmentation, and bioassimilation by a Comamonas sp. (FIG. 1B) Cell growth and MPs release with (top) PET films as the sole carbon source, (middle) with 5 mM acetate as a co-substrate, or (bottom) with 20 mM acetate as a co-substrate. Control represents cells grown on the two respective acetate concentrations without the PET film. (FIG. 1C) Cell growth and MPs release with (left) PET pellets as the sole carbon source, (middle) with 5 mM acetate as a co-substrate, or (right) with 20 mM acetate as a co-substrate. Control represents cells grown on the two respective acetate concentrations without the PET pellet. (FIG. 1D) SEM images of washed PET films from the following incubation conditions: (top) growth medium with no cells, (middle) growth medium with cells, and (bottom) growth medium with cells supplemented with 5 mM acetate. (FIG. 1E) SEM images of washed PET pellets from the following incubation conditions: (top) growth medium with no cells, (middle) growth medium with cells, and (bottom) growth medium with cells supplemented with 5 mM acetate. For (FIG. 1B) and (FIG. 1C), error bars represent the standard deviation of the mean of three biological replicates and two-tailed t-test was performed on the last data points: *P<0.05, **P<0.01, ***P<0.001, ns: not significant.

FIG. 2A-2C. Production of nanoplastic particles from PET pellet fragmentation by C. testosteroni. (FIG. 2A) TEM images of suspensions after incubations of C. testosteroni with PET pellets and 5 mM acetate (Ac) as a co-substrate for (left) 1 d and (right) 30 d. (FIG. 2B) Nanoparticle size concentration and (FIG. 2C) violin plot with inserted box plot of particle size distribution following 1 d, 15 d, and 30 d of PET pellets incubated with cells and 5 mM Ac. In FIG. 2B, the solid lines represent the mean value of the particle concentration, and the shades represent the 95% confidential intervals. In FIG. 2A, White arrows indicate NPs. In FIG. 2C, the top and bottom edges of the box plot represent the first and third quartiles, respectively, the line inside represents the median value, and the whiskers represent the upper and lower extremes in the data. The width of the violin plot illustrates the concentration of different particle sizes. In FIG. 2C, Welch's t-test was performed for pairwise comparison due to the unequal sample sizes and variances in different conditions (*P<0.05, **P<0.01, ***P<0.001). Data from an independent duplicate measurement for B and C are shown in FIGS. 11A and 11B.

FIGS. 3A and 3B. Probing modifications of the surface chemistry of PET films and pellets from biofragmentation by C. testosteroni. Changes in normalized FTIR intensity for different functional groups in (A) PET films (n=5) after 27-d incubation and (B) PET pellets (n=6) after 42-d incubation in the following conditions: with the nutrient medium with no cells (control), with cells in the nutrient medium (+cells), with cells in the nutrient medium supplemented with 5 mM acetate (+cells+Ac). In (FIG. 3A) and (FIG. 3B), one-way ANOVA analysis with post hoc Tukey's test was performed: *P<0.05, **P<0.01, ***P<0.001, ns: not significant.

FIGS. 4A-4E. Production of bioavailable monomer from hydrolysis of PET oligomer by C. testosteroni. (FIG. 4A) UHPLC spectrum of the quantification of BHET, a PET-related oligomer, and its ester hydrolysis products, MHET and TPA. (FIG. 4B) Tracking the depletion of 2.5 mM BHET and corresponding production of MHET and TPA in (top) the absence and (bottom) the presence of C. testosteroni KF-1 cells. (FIG. 4C) Fold change in biomass growth from starting cell abundance after 24 h of growth on 2.5 mM BHET alone, a mixture of 2.5 mM BHET and 5 mM acetate (Ac), or 5 mM Ac. (FIG. 4D) (top) Depletion of TPA and (bottom) associated biomass growth with C. testosteroni KF-1 grown on 3.75 mM TPA as the only carbon source. (FIG. 4E) Schematic illustration of biotransformation of PET-related oligomer to an assimilable PET monomer by C. testosteroni KF-1. In (FIG. 4C), two-tailed 1-test was performed: *P<0.05, **P<0.01, ***P<0.001.

FIGS. 5A-5F. Proteomics analysis and protein homology modeling of candidate hydrolases in C. testosteroni. (FIG. 5A) Total spectra count identified from intracellular intracellular proteomics of C. testosteroni KF-1. (FIG. 5B) Venn diagrams of the proteins identified in each condition from intracellular proteomics. (FIG. 5C) Volcano plots of adjusted P value for significance (y axis) versus log 2 fold change (x axis) in the abundance of proteins in (top) cells fed on PET relative to cells fed on Ac alone or (bottom) cells fed on both PET and acetate relative cells on acetate alone. (FIG. 5D) Proteomics profiling of hydrolases encoded in the genome of C. testosteroni KF-1. (FIG. 5E) Homology modeling of the identified hydrolase (CtesDRAFT_PD1902) and shown in color are several binding site motifs conserved in esterase-type hydrolases with reported PET activity: catalytic residues (Ser158, Asp253, and His283; red); the ⁸⁵HGGG⁸⁸ motif (blue), two esterase catalytic motifs, 156GXSXG¹⁶⁰ in orange, and ²⁵³DPXXD²⁵⁷ in yellow. (FIG. 5F) Optimized binding of BHET in the predicted binding site of the modeled hydrolase structure. In FIG. 5A, FIG. 5B, and FIG. 5D, data were obtained from four biological replicates. In (FIG. 5A), two-tailed t-test was performed: *P<0.05, **P<0.01, ***P<0.001. In (FIG. 5C), significant difference in protein abundances (|log 2 fold change|>1 and P<0.05) are indicated in red (higher abundance) and blue (lower abundance); gray dots represent genes without significant changes (NS) in protein levels. In (FIG. 5C), selected proteins are labeled: aconitate hydratase (Acn); citrate synthase (GltA); succinate dehydrogenase (Sdh); flagellin protein (FliC); copper resistance protein (CopC); a gene encoding an organic hydroperoxide resistance protein (CtesDRAFT_PD0278). In (FIG. 5F), amino acid residues are labeled with their three-letter codes and sequence numbers; key atoms are noted in purple. Important distances between BHET and catalytic residues are shown in green.

FIGS. 6A-6E. Fragmentation of PET polymer and hydrolysis of PET oligomer by cell-free secretions from C. testosteroni KF-1. (FIG. 6A) Schematic illustration of transformation of BHET by cell-free secretions of C. testosteroni KF-1. (FIG. 6B) Depletion of BHET and associated production of MHET and TPA during reaction of BHET with cell-free spent media of 5 mM acetate-grown C. testosteroni KF-1. (FIG. 6C) Schematic illustration of biofragmentation of PET plastics by cell-free secretions from spent media of acetate-grown C. testosteroni KF-1. (FIG. 6D) Release of MPs from (left) PET films 389 and (right) PET pellets incubated with cell-free spent media obtained after growth of C. testosteroni KF-1 on 5 mM or 20 mM of acetate. Control represents PET incubated with nutrient media without cells or acetate. (FIG. 6E) (top) Schematic illustration of heterologous expression of TPA utilization and CtesDRAFT_PD1902 genes in P. putida KT2440 and (bottom) percentage of BHET hydrolysis to TPA when incubated with the different strains in the presence of 5 mM acetate as a co-substrate. In (FIG. 6B) and (FIG. 6D), error bars represent the standard deviation of the mean of three replicates. In FIG. 6D, two-tailed t-tests were performed to compare 20 mM Ac and 5 mM Ac to the control. In (FIG. 6E), one-way ANOVA analysis with post hoc Tukey's test was performed. *P<0.05, **P<0.01, ***P<0.001, ns: not significant.

FIG. 7. Schematic Overview of the Plastics Degradation Traits of Wastewater Comamonas. The data presented in this study provide evidence biodeterioration and biofragmentation of PET materials, hydrolysis of PET oligomer, and assimilation of PET monomer by C. testosteroni.

FIGS. 8A-8C. (FIG. 8A) Schematic illustration of genetic engineering to delete CtesDRAFT_PD1902 and re-introduce the gene back to C. testosteroni after the deletion. (FIG. 8B) Percentage of BHET conversion when incubated with C. testosteroni wild-type and mutant strains in the presence of 5 mM acetate as a co-substrate for 48 hours. (FIG. 8C) Final optical density (OD₆₀₀) from PET pellets when incubated with C. testosteroni wild-type and mutant strains in the presence of 5 mM acetate as a co-substrate for 19 days.

FIGS. 9A-9C. SEM images of the reference (FIG. 9A) PET films and (FIG. 9B) PET pellets; scale bars specify the magnification for each image. (FIG. 9C) superimposed FTIR spectra for the reference PET pellet (red) and a reference PET film (back).

FIG. 10. Weight loss of PET films and pellets after incubating with C. testosteroni KF-1. One-way ANOVA analysis with post hoc Tukey's test was performed: *P<0.05, **P<0.01, ***P<0.001. ns: not significant.

FIGS. 11A and 11B. (FIG. 11A) Nanoparticle size concentration and (FIG. 11B) violin plot with inserted box plot of particle size distribution following 1 d, 15 d, and 30 d of PET pellets incubated with cells and 5 mM Ac. In FIG. 11A, the solid lines represent the mean value of the particle concentration, and the shades represent the 95% confidential intervals. In FIG. 11B, the top and bottom edges of the box plot represent the first and third quartiles, respectively, the line inside represents the median value, and the whiskers represent the upper and lower extremes in the data. The width of the violin plot illustrates the concentration of different particle sizes. In (FIG. 11B), Welch's t-test was performed for pairwise comparison due to the unequal sample sizes and variances in different conditions (*P<0.05, **P<0.01, ***P<0.001). Data was collected from one biological replicate at each incubation time.

FIGS. 12A and 12B. FTIR spectra revealed functional group change on the surface of PET films (FIG. 12A) and pellets (FIG. 12B) after incubating with C. testosteroni KF-1.

FIGS. 13A and 13B. (FIG. 13A) Cell growth of C. testosteroni KF-1 on MHET. (FIG. 13B) MHET consumption during the incubation.

FIGS. 14A and 14B. (FIG. 14A) Schematic illustration (left) and release of MPs from PET films (middle) and PET pellets (right) incubated with cell-free spent media obtained after growth of C. testosteroni KF-1 on 5 mM or 20 mM of acetate. (FIG. 14B) Schematic illustration (left) and depletion of BHET (middle) and associated production of MHET and TPA (right) during reaction of BHET with cell-free spent media of 5 mM acetate-grown C. testosteroni KF-1.

FIG. 15. Total spectra identified from intracellular and extracellular proteomics of C. testosteroni KF-1.

### DETAILED DESCRIPTION

This disclosure presents engineered bacteria, systems, and methods for degrading polyester materials, including materials having polyethylene terephthalate (PET). An aspect is an engineered bacterial strain that can include one or more heterologous hydrolase capable of degrading at least a portion of a polyester material. In aspects, the hydrolase can be derived from, or native to, a bacterium in the Comamonadacae family. In one or more aspects, the hydrolase can be from Comamonas testosteroni. In aspects, the engineered bacterial strain is Pseudomonas putida, Cupriavidus necator, Corynebacterium glutamicum, Zymomonas mobilis, Rhodococcus jostii, and/or Bacillus licheniformis.

In the aspects, the hydrolases are capable of depolymerizing and/or hydrolizing at least a portion of a polyester material, e.g., a material comprising polyethylene terephthalate (PET). In certain aspects, the hydrolase can hydrolize bis(2-hydroxyethyl) terephthalate (BHET) and/or a material comprising BHET and/or PET.

Another aspect is a method of fragmentation and/or degradation of at least a portion of a polyester material. The method may include exposing the polyester material to one or more hydrolase from a bacterium in the Comamonadacae family. In one or more aspects, the hydrolase can be from Comamonas testosteroni.

### Definitions and Terminology

The disclosed engineered bacteria, systems, and methods for degrading polyester materials, e.g., materials comprising polyethylene terephthalate (PET), may be further described using definitions and terminology as follows. The definitions and terminology used herein are for the purpose of describing particular embodiments only and are not intended to be limiting.

As used in this specification and the claims, the singular forms “a,” “an,” and “the” include plural forms unless the context clearly dictates otherwise. For example, the term “a polynucleotide” or an “allergen protein” should be interpreted to mean “one or more polynucleotides” and “one or more allergen proteins,” respectively, unless the context clearly dictates otherwise. As used herein, the term “plurality” means “two or more.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or “B” or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

### Polynucleotides

The terms “nucleic acid” and “oligonucleotide,” as used herein, refer to polydeoxyribonucleotides (containing 2-deoxy-D-ribose), polyribonucleotides (containing D-ribose), and to any other type of polynucleotide that is an N glycoside of a purine or pyrimidine base. There is no intended distinction in length between the terms “nucleic acid”, “oligonucleotide” and “polynucleotide”, and these terms will be used interchangeably. These terms refer only to the primary structure of the molecule. Thus, these terms include double- and single-stranded DNA, as well as double- and single-stranded RNA.

Oligonucleotides can be prepared by any suitable method, including direct chemical synthesis by a method such as the phosphotriester method of Narang et al., 1979, Meth. Enzymol. 68:90-99; the phosphodiester method of Brown et al., 1979, Meth. Enzymol. 68:109-151; the diethylphosphoramidite method of Beaucage et al., 1981, Tetrahedron Letters 22:1859-1862; and the solid support method of U.S. Pat. No. 4,458,066, each incorporated herein by reference. A review of synthesis methods of conjugates of oligonucleotides and modified nucleotides is provided in Goodchild, 1990, Bioconjugate Chemistry 1 (3): 165-187, incorporated herein by reference.

The term “promoter” refers to a cis-acting DNA sequence that directs RNA polymerase and other trans-acting transcription factors to initiate RNA transcription from the DNA template that includes the cis-acting DNA sequence.

The polynucleotide sequences contemplated herein may be present in expression vectors. For example, the vectors may comprise a polynucleotide encoding a protein. The polynucleotide present in the vector may be operably linked to a promoter. “Operably linked” refers to the situation in which a first nucleic acid sequence is placed in a functional relationship with a second nucleic acid sequence. For instance, a promoter is operably linked to a coding sequence if the promoter affects the transcription or expression of the coding sequence. Operably linked DNA sequences may be in close proximity or contiguous and, where necessary to join two protein coding regions, in the same reading frame. Vectors contemplated herein may comprise a heterologous promoter (e.g., a eukaryotic or prokaryotic promoter) operably linked to a polynucleotide that encodes a protein. A “heterologous promoter” refers to a promoter that is not the native or endogenous promoter for the protein or RNA that is being expressed. Vectors as disclosed herein may include plasmid vectors.

The terms “polynucleotide,” “polynucleotide sequence,” “nucleic acid” and “nucleic acid sequence” refer to a nucleotide, oligonucleotide, polynucleotide (which terms may be used interchangeably), or any fragment thereof. These phrases also refer to DNA or RNA of genomic, natural, or synthetic origin (which may be single-stranded or double-stranded and may represent the sense or the antisense strand).

Regarding polynucleotide sequences, the terms “percent identity” and “% identity” refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences. Percent identity for a nucleic acid sequence may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST), which is available from several sources, including the NCBI, Bethesda, Md., at its website. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at the NCBI website. The “BLAST 2 Sequences” tool can be used for both blastn and blastp (discussed above).

Regarding polynucleotide sequences, percent identity may be measured over the length of an entire defined polynucleotide sequence, for example, as defined by a particular SEQ ID number, or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length supported by the sequences shown herein, in the tables, figures, or Sequence Listing, may be used to describe a length over which percentage identity may be measured.

Regarding polynucleotide sequences, “variant,” “mutant,” or “derivative” may be defined as a nucleic acid sequence having at least 50% sequence identity to the particular nucleic acid sequence over a certain length of one of the nucleic acid sequences using blastn with the “BLAST 2 Sequences” tool available at the National Center for Biotechnology Information's website. (See Tatiana A. Tatusova, Thomas L. Madden (1999), “Blast 2 sequences-a new tool for comparing protein and nucleotide sequences”, FEMS Microbiol Lett. 174:247-250). Such a pair of nucleic acids may show, for example, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity over a certain defined length.

Nucleic acid sequences that do not show a high degree of identity may nevertheless encode similar amino acid sequences due to the degeneracy of the genetic code where multiple codons may encode for a single amino acid. It is understood that changes in a nucleic acid sequence can be made using this degeneracy to produce multiple nucleic acid sequences that all encode substantially the same protein. For example, polynucleotide sequences as contemplated herein may encode a protein and may be codon-optimized for expression in a particular host.

A “recombinant nucleic acid” is a sequence that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques known in the art. The term recombinant includes nucleic acids that have been altered solely by addition, substitution, or deletion of a portion of the nucleic acid. Frequently, a recombinant nucleic acid may include a nucleic acid sequence operably linked to a promoter sequence. Such a recombinant nucleic acid may be part of a vector that is used, for example, to transform a cell.

### Peptides, Polypeptides, and Proteins

As used herein, the terms “peptide,” “polypeptide,” and “protein,” refer to molecules comprising a chain a polymer of amino acid residues joined by amide linkages. The term “amino acid residue,” includes but is not limited to amino acid residues contained in the group consisting of alanine (Ala or A), cysteine (Cys or C), aspartic acid (Asp or D), glutamic acid (Glu or E), phenylalanine (Phe or F), glycine (Gly or G), histidine (His or H), isoleucine (Ile or I), lysine (Lys or K), leucine (Leu or L), methionine (Met or M), asparagine (Asn or N), proline (Pro or P), glutamine (Gln or Q), arginine (Arg or R), serine (Ser or S), threonine (Thr or T), valine (Val or V), tryptophan (Trp or W), and tyrosine (Tyr or Y) residues. The term “amino acid residue” also may include nonstandard or unnatural amino acids. The term “amino acid residue” may include alpha-, beta-, gamma-, and delta-amino acids.

In some embodiments, the term “amino acid residue” may include nonstandard or unnatural amino acid residues. The term “amino acid residue” may include L isomers or D isomers of any of the aforementioned amino acids.

As used herein, a “peptide” is defined as a short polymer of amino acids, of a length typically of 20 or less amino acids, and more typically of a length of 12 or less amino acids (Garrett & Grisham, Biochemistry, 2nd edition, 1999, Brooks/Cole, 110). In some embodiments, a peptide as contemplated herein may include no more than about 2, 3, 4, 5, 6, 7, 8, 9, 10, 11, 12, 13, 14, 15, 16, 17, 18, 19, or 20 amino acids. A polypeptide, also referred to as a protein, is typically of length >100 amino acids (Garrett & Grisham, Biochemistry, 2nd edition, 1999, Brooks/Cole, 110). A polypeptide, as contemplated herein, may comprise, but is not limited to, 100, 101, 102, 103, 104, 105, about 110, about 120, about 130, about 140, about 150, about 160, about 170, about 180, about 190, about 200, about 210, about 220, about 230, about 240, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, about 1000, about 1100, about 1200, about 1300, about 1400, about 1500, about 1750, about 2000, about 2250, about 2500 or more amino acid residues.

Regarding proteins, the phrases “percent identity” and “% identity,” refer to the percentage of residue matches between at least two amino acid sequences aligned using a standardized algorithm. Methods of amino acid sequence alignment are well-known. Some alignment methods take into account conservative amino acid substitutions. Such conservative substitutions, explained in more detail below, generally preserve the charge and hydrophobicity at the site of substitution, thus preserving the structure (and therefore function) of the polypeptide. Percent identity for amino acid sequences may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST), which is available from several sources, including the NCBI, Bethesda, Md., at its website. The BLAST software suite includes various sequence analysis programs including “blastp,” that is used to align a known amino acid sequence with other amino acids sequences from a variety of databases.

Regarding proteins, percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are provided as an example.

Regarding proteins, the amino acid sequences of variants, mutants, or derivatives as contemplated herein may include conservative amino acid substitutions relative to a reference amino acid sequence. For example, a variant, mutant, or derivative protein may include conservative amino acid substitutions relative to a reference molecule. “Conservative amino acid substitutions” are those substitutions that are a substitution of an amino acid for a different amino acid where the substitution is predicted to interfere least with the properties of the reference polypeptide. In other words, conservative amino acid substitutions substantially conserve the structure and the function of the reference polypeptide. The following provides a list of exemplary conservative amino acid substitutions which are contemplated herein.

| Original | |
|---|---|
| Residue | Conservative Substitution |
| Ala | Gly, Ser |
| Arg | His, Lys |
| Asn | Asp, Gln, His |
| Asp | Asn, Glu |
| Cys | Ala, Ser |
| Gln | Asn, Glu, His |
| Glu | Asp, Gln, His |
| Gly | Ala |
| His | Asn, Arg, Gln, Glu |
| Ile | Leu, Val |
| Leu | Ile, Val |
| Lys | Arg, Gln, Glu |
| Met | Leu, Ile |
| Phe | His, Met, Leu, Trp, Tyr |
| Ser | Cys, Thr |
| Thr | Ser, Val |
| Trp | Phe, Tyr |
| Tyr | His, Phe, Trp |
| Val | Ile, Leu, Thr |

Conservative amino acid substitutions generally maintain (a) the structure of the polypeptide backbone in the area of the substitution, for example, as a beta sheet or alpha helical conformation, (b) the charge or hydrophobicity of the molecule at the site of the substitution, and/or (c) the bulk of the side chain. Non-conservative amino acids typically disrupt (a) the structure of the polypeptide backbone in the area of the substitution, for example, as a beta sheet or alpha helical conformation, (b) the charge or hydrophobicity of the molecule at the site of the substitution, and/or (c) the bulk of the side chain.

### Hydrolases

In various aspects, one or more hydrolases are disclosed. The hydrolases are capable of depolymerizing and/or hydrolizing at least a portion of a polyester material, e.g., a material comprising polyethylene terephthalate (PET). In certain aspects, the hydrolase can hydrolize bis(2-hydroxyethyl) terephthalate (BHET) and/or a material comprising BHET and/or PET. In various aspects, the hydrolase can be derived from, or native to, a bacterium in the Comamonadacae family. In one or more aspects, the hydrolase can be from Comamonas testosteroni. In the same or alternative aspects, the hydrolase can be from Comamonas testosteroni KF-1. In certain aspects, the hydrolase can be CtesDRAFT_PD1902 and/or CtesDRAFT_PD3135.

In various aspects, the hydrolase can comprise, consist essentially of, or consist of an amino acid sequence that is at least 80%, at least 85%, at least 88%, at least 90%, at least 95%, at least 99%, or 100% identical to SEQ ID NO: 1. In one or more aspects, a polynucleotide is provided that encodes for an amino acid sequence that is at least 80%, at least 85%, at least 88%, at least 90%, at least 95%, at least 99%, or 100% identical to SEQ ID NO: 1.

In various aspects, the hydrolase can be encoded by a polynucleotide, e.g., an expression plasmid, having a polynucleotide sequence that is at least 80%, at least 85%, at least 88%, at least 90%, at least 95%, at least 99%, or 100% identical to SEQ ID NO: 2.

### Engineered Bacteria

In various aspects, engineered bacteria are disclosed. In one or more aspects, the engineered bacteria can comprise one or more of the hydrolases described herein. In certain aspects, the hydrolase is a heterologous hydrolase. In various aspects, the one or more heterologous hydrolase is from Comamonas testosteroni. In various aspects, the one or more heterologous hydrolase is CtesDRAFT_PD1902 and/or CtesDRAFT_PD3135.

In aspects, the engineered bacteria can be engineered to express the heterologous hydrolase. In certain aspects, the engineered bacteria can be an engineered Pseudomonas putida (P. putida), Cupriavidus necator (C. necator), Corynebacterium glutamicum (C. glutamicum), Zymomonas mobilis (Z. mobilis), Rhodococcus jostii (R. jostii), and/or Bacillus licheniformis (B. licheniformis.)

### Methods

In various aspects, methods are disclosed for the degradation and/or fragmentation of a polyester material or of a material comprising a polyester. In various aspects, the method can include exposing a polyester material to one or more hydrolase, for example from the Comamonadacae family. In such aspects, the hydrolase can be derived from, or native to, Comamonas testosteroni. In various aspects, the hydrolase can be any of the hydrolases described herein. In certain aspects, the methods can include exposing the material comprising a polyester to an engineered bacteria that comprises the hydrolase. In such aspects, any of the engineered bacteria described herein may be used.

In one or more aspects, the polyester material can be any material that comprises a polyester. In certain aspects, the polyester material can be any material that comprises PET. In certain aspects, the material may be bis(2-hydroxyethyl) terephthalate (BHET) and/or a material comprising BHET and/or PET.

In certain aspects, the hydrolase can hydrolize bis(2-hydroxyethyl) terephthalate (BHET) and/or a material comprising BHET and/or PET.

In certain aspects, the methods can include exposing a culture of any of the engineered bacteria described herein to a waste and/or recycling stream that includes materials comprising a polyester, e.g., PET.

### EXAMPLES

The following Examples are illustrative and are not intended to limit the scope of the invention.

### Example 1—Mechanisms of Polyethylene Terephthalate Pellet Fragmentation into Nanoplastics and Assimilable Carbons by Wastewater Comamonas

Comamonadacae family of bacteria are enriched on polyethylene terephthalate (PET) microplastics in wastewaters and urban rivers. Here, the plastics-degrading mechanisms employed by Comamonas testosteroni KF-1, a wastewater isolate, to grow on PET films or pellets were investigate. In accordance with 8-fold higher optical density with the pellets than with the films, scanning electron microscopy shows significant fragmentation of the pellets but only minor dents on the films, indicating a dependence of biodeterioration on the plastics morphology. Nanoparticle tracking determines a 3.5-fold increase in the relative abundance of small nanoparticles (<100-nm diameter) during a 30-day cultivation with the pellets; infrared spectroscopy reveals that this biofragmentation is due to hydrolytic rather than oxidative cleavage. Liquid chromatography analysis of culture solutions demonstrates double hydrolysis of a PET oligomer, bis-(2-hydroxyethyl) terephthalate, to the bioavailable monomer terephthalate. Supplementation of cultures with acetate, a common wastewater co-substrate, promotes both PET biofragmentation and cell growth. Further, Comamonas testosteroni cells fed on only acetate produce secretions with activity for catalytic hydrolysis of PET polymer and oligomer. Of the multiple hydrolases encoded in the bacterial genome, intracellular proteomics identifies only one, which is found in both PET-only and acetate-only conditions. Protein homology modeling of this hydrolase structure illustrates substrate binding analogous to reported PET hydrolases, despite dissimilar sequence identity. Implementation of the Comamonas hydrolase gene in Pseudomonas putida, a biotechnologically relevant bacterium that natively lacks PET-degrading capability, enables hydrolysis of a PET oligomer. Thus, wastewater Comamonas species constitutively exhibit traits that can be exploited for engineered plastics bioconversion.

### Significance

Microbes with native capabilities for plastics biodegradation are of particular interest due to the recalcitrance of plastics materials. Bacteria from the Comamonadacae family are predominant on polyethylene terepthalate (PET) microplastics in various environments, but the degradation mechanisms by these bacteria remain unknown. Here how a wastewater Comamonas isolate affords to grow on PET materials is investigated by combining microscopy, spectroscopy, proteomics, protein modeling, and genetic engineering. First, the mechanism of biofragmentation into nanoplastics is unraveled. Second, a key hydrolase, which is not a putative PET hydrolase, is identified as responsible for PET depolymerization into bioavailable carbons. While wastewater Comamonas can produce potentially harmful plastic nanoparticles, they exhibit capabilities for complete degradation of PET plastics as attractive traits for biotechnology.

### Introduction

Ubiquitous and extensive usage of plastic materials has led to the accumulation of plastic wastes, which are projected to reach ˜33 billion tons by the year 2050 (1). Plastic wastes have been reported in various environments including marine (2, 3) and fresh waters (4, 5), sediments (6, 7), and soils (8, 9). Microplastics (MPs) and nanoplastics (NPs), defined operationally as plastic fragments with sizes smaller than 5 mm and 1 μm, respectively, are considered a threat to both aquatic and terrestrial ecosystems, as organisms can easily ingest these small particles (9-13). Since plastic wastes are entirely anthropogenic, wastewater treatment plants (WWTP) represent important repositories for plastics (14-18), contributing to their release into natural systems and serving as a source of MPs and NPs (19, 20). Wastewater effluents contain various types of MPs, among which polyethylene terephthalate (PET) MPs are the most abundant, constituting approximately 50% of MPs in the effluents (14). In fact, as an extensively used polymer in disposable containers, PET accounts for 12% of global solid waste (21). Thus, there is increased research interest focusing on the fate of PET, especially within the context of biodeterioration and biodegradation of PET plastic materials by WWTP-associated microorganisms.

It is well documented that microorganisms can generate enzymes to deteriorate and modify the surface of PET plastics, facilitating the release of PET MPs and carbon derivatives to be used as carbon sources to support microbial growth (22, 23). Most previous research has focused on microbial consortia (24-28) or a few fungal and bacterial isolates with PET hydrolases, also termed PETases (29-32). Of particular interest are the mechanisms through which microorganisms enriched in wastewater sludge can achieve biofragmentation of PET plastics to release MPs, NPs, and eventually the monomer compounds of PET.

Bacteria belonging to the family Comamonadaceae have been found to be predominant on MPs that are present in WWTP effluents and urban rivers (33-35). Furthermore, colonization of seven different MPs, including PET MPs, with WWTP effluent water consistently led to enriched abundance of the family Comamonadaceae compared to the relative distribution of these bacteria in the effluent water. (36) Importantly, as one of the most enriched genera in wastewater sludge, (37, 38) the genus Comamonas of the family Comamonadaceae contains several species with the ability to catabolize a wide spectrum of aromatic compounds (39-42). including a monomer derivative of PET (43, 44). However, it is not yet known whether wastewater Comamonas species possess PET-degrading enzymes to facilitate the depolymerization of PET plastics.

Comamonas testosteroni KF-1, which was isolated from a sewage sludge enrichment as a bacterium that can degrade laundry surfactants (45), was recently shown to metabolize terephthalate (TPA), a monomer of the PET polymer (44). Wilkes et al. (44) elucidated the assimilation route of TPA in C. testosteroni KF-1 through the 4,5-meta cleavage pathway to yield oxaloacetate and pyruvate, two intermediates in the central carbon metabolism. In addition, the genome of C. testosteroni KF-1 encodes for multiple hydrolases (45), which could participate potentially in plastic depolymerization, as demonstrated previously in other microorganisms (46-50). Yet, it has not yet been demonstrated that C. testosteroni and related wastewater Comamonas species produce enzymes with PET-hydrolase activity to facilitate the biofragmentation of PET materials (51).

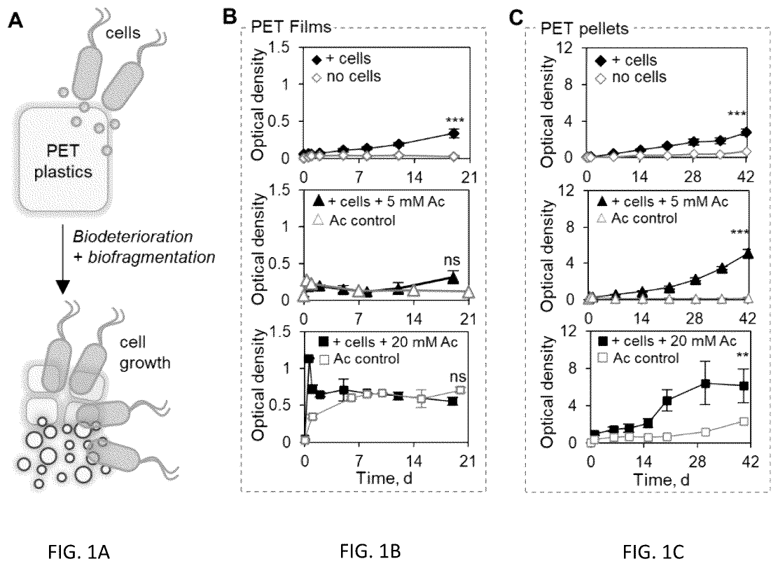

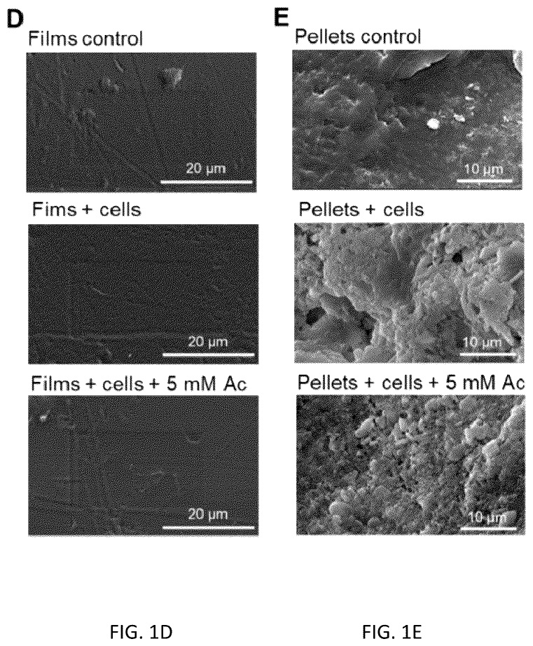

Given prior evidence of colonization of PET plastics by bacteria of the Comamonadacae family (33-35), we hypothesize that the genome-encoded hydrolases in C. testosteroni KF-1 are capable of deteriorating and fragmenting PET plastics, generating bioavailable breakdown products to support bacterial growth (FIG. 1A). This hypothesis was tested with PET films and PET pellets incubated with C. testosteroni KF-1, and assessed PET biodegradation mechanisms with a suite of orthogonal techniques. First, bacterial cell growth and formation of MPs by optical density was monitored. Second, surface deconstruction and particle morphology of the PET films and pellets was visualized by scanning electron microscopy (SEM) and transmission electron microscopy (TEM). Third, the concentration and sizes of NPs was tracked by nanoparticle tracking analysis. Fourth, the mechanisms of plastic surface modification was examined by attenuated total reflectance-Fourier transform infrared (ATR-FTIR) spectroscopy and confirmed this mechanism by monitoring degradation products of PET pellets and PET-related oligomer using ultra high-performance liquid chromatography (UHPLC). Fifth, proteomics data was obtained to identify hydrolase enzymes, followed by molecular docking simulations to explore binding mechanisms informed by previous studies with known PETase structures. Sixth, the Comamonas gene that encodes an identified hydrolase from the proteomics data was inserted into Pseudomonas putida KT2440, widely considered a bacterial chassis for waste carbon conversion but lacking PET-degrading capability (52, 53), and subsequently determined the hydrolytic activity of the constructed mutant towards a PET oligomer. The findings presented herein shed light on the mechanisms that afford the growth of wastewater Comamonas on PET microplastics and advance the consideration of C. testosteroni KF-1 as a promising biocatalytic platform for plastics conversion in engineered waste carbon recycling.

### Results

Bacterial Growth and MPs Release from PET Plastics.

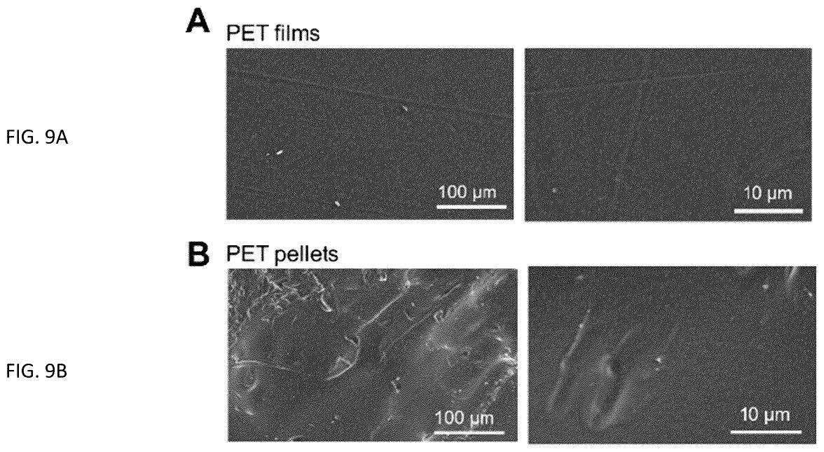

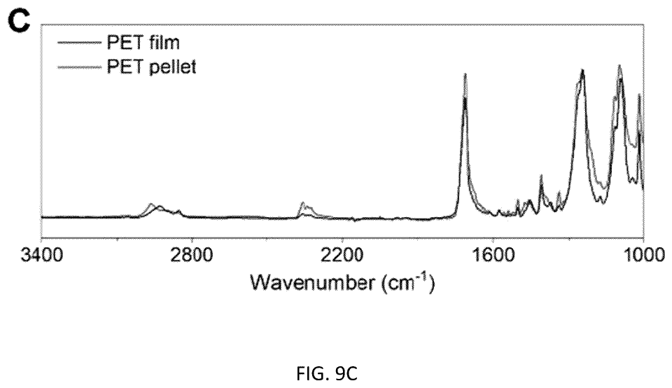

The initial morphology and surface chemistry of the starting PET plastics used in the experiments was probed using SEM and FTIR, respectively (FIGS. 9A-9C). While the PET films exhibit smooth surfaces, the surfaces of the PET pellets were irregular with noticeable indents (FIGS. 9A-9C). Despite the different surface morphologies, both PET films and pellets exhibited similar surface chemistry as determined by the vibrational bands of functional groups captured by FTIR spectroscopy (FIGS. 9A-9C). We hypothesize that the morphological differences would affect cell surface attachment and thus influence the extent of the plastics degradation (50). We monitored the optical density at 600 nm (OD₆₀₀) as a function of exposure time of the C. testosteroni cells to the PET films and pellets (FIG. 1B, 1C). We obtained 8-fold to 16-fold higher optical density values (P<0.001) when the PET films or pellets were exposed alone to the C. testosteroni KF-1 cells compared to the control experiment in the absence of cells (FIG. 1B, IC). Due to some increase of OD₆₀₀ intensity caused by minor deterioration of the PET pellets and films to MPs by the nutrient media in the absence of cells, we attributed the increase in OD₆₀₀ values in the presence of cells to both cell growth and the fragmentation and release of MPs.

Experiments were also conducted with the PET materials co-incubated with acetate, a common volatile fatty acid widely found in WWTP (54-57). When acetate was present as a co-substrate with the PET, up to 25-fold higher OD₆₀₀ values were obtained than with cells grown on acetate alone (P<0.001), either initially in the case of the PET films or throughout the entire experiments in the case of the PET pellets (FIG. 1B, IC). Therefore, the presence of an additional carbon source as would be expected in wastewaters would lead to enhanced biofragmentation of PET plastics, promoting the release of MPs. Next, using SEM and TEM, we probed changes in the polymer surface morphology that would be a consequence of the observed degradation of the PET plastics.

### PET Plastic Surface Modification Due to Biodeterioration and Biofragmentation.

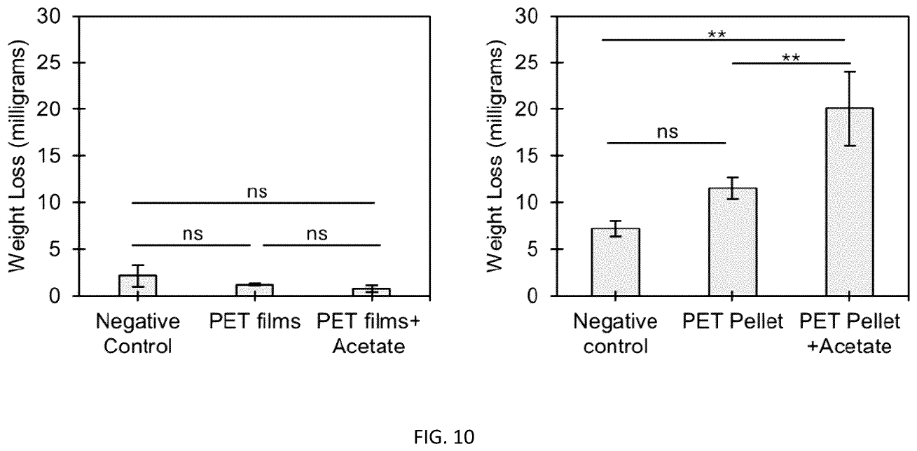

The deterioration and fragmentation of PET plastics following microbial colonization are expected to alter the morphology of the plastic surface (27, 58). To visualize this morphological alteration, we performed SEM on PET films and pellets after incubating at different conditions without and with the C. testosteroni KF-1 cells (FIG. 1D, 1E). The surface of the PET films only exhibited very minor changes characterized by some indents on the film surface when acetate was present as a co-substrate; in the absence of acetate in the medium, there were minimal to no alterations of PET film surface with or without the bacterial cells (FIG. 1D). In contrast, the incubation of the PET pellets with the bacterial cells led to an irregular rough surface of the PET pellets with several etched areas; the addition of acetate as an exogenous carbon source enhanced these morphological changes accompanied by deep etches and pitting throughout the surface of the PET pellets (FIG. 1E). The PET pellets exposed to the nutrient medium without bacterial cells, however, maintained a smooth-like surface, confirming that the bacterial cells were responsible for the observed changes in the PET surface morphology (FIG. 1E). Therefore, the extent of surface modification illustrated by the SEM images of the PET films and PET pellets was consistent with the different extent of biofragmentation implied by the OD₆₀₀ and plastic weight loss measurements whereby there were up to one order of magnitude higher OD₆₀₀ values and more weight loss with the PET pellets compared to the PET films (FIG. 1B, 1C; SI Appendix, FIG. 10).

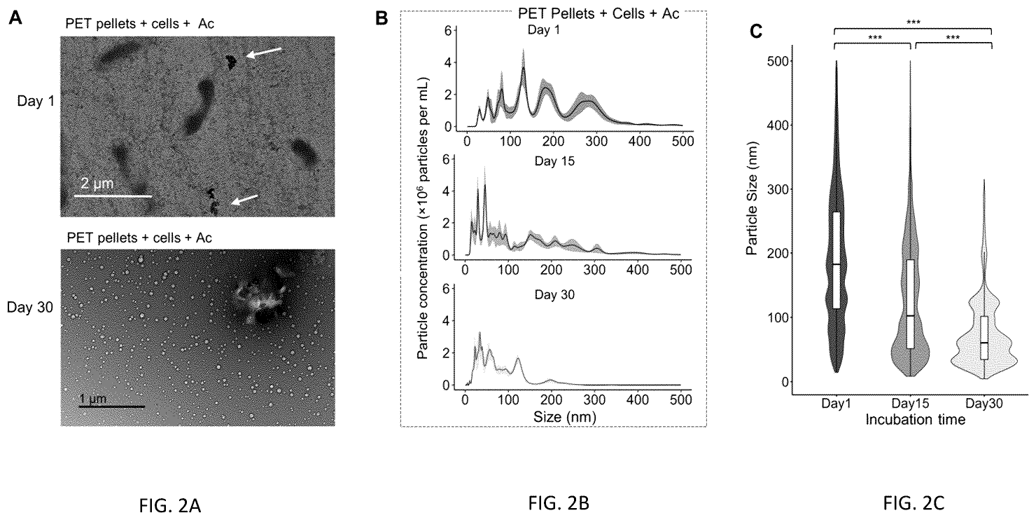

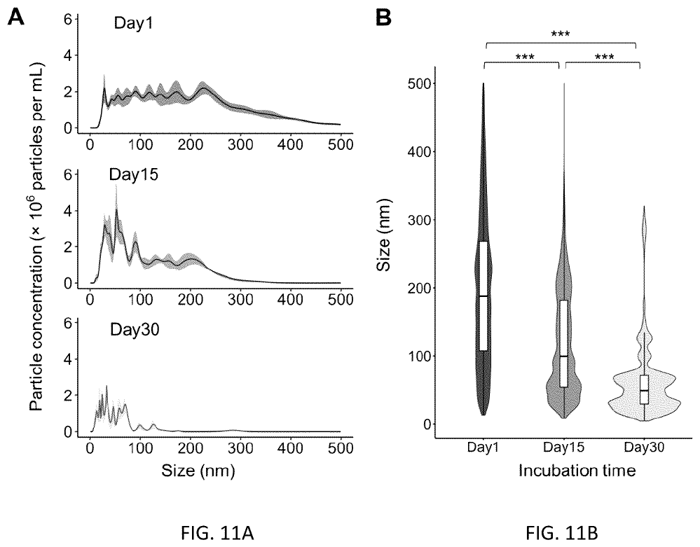

Due to the elevated OD₆₀₀ values indicating MP release and the SEM images illustrating extensive biodeterioration of the surface of the PET pellets in the presence of C. testosteroni KF-1 cells and acetate (FIG. 1D, 1E), we probed for the possible production of both large NPs (100-500 nm) and small NPs (less than 100 nm) from the PET pellets (FIG. 2). The TEM images illustrated the evolution of large-sized to small-sized nanoparticles from 1 d to 30 d after incubation of the PET pellets (FIG. 2A). For quantitative corroboration of the TEM images, we conducted nanoparticle tracking analysis to quantify the distribution of the particles (FIG. 2B, 2C; FIGS. 11A and 11B). The data revealed that the majority of particles (79%) was larger than 100 nm after 1 day of incubation, but the particle size was subsequently decreased as a function of incubation time with the bacterial cells (FIG. 2B). Specifically, the proportion of the particles smaller than 100 nm increased from 21% on day 1 to 49% on day 15 and to 74% on day 30 (FIG. 2C; FIGS. 11A and 11B). To explore the mechanisms underlying the PET biofragmentation, we performed spectroscopic analysis via ATR-FTIR to reveal the surface chemistry of the PET pellets and subsequently analyzed the products generated from a PET oligomer via UHPLC.

### Production of Assimilable Carbon Products Via Hydrolytic Biofragmentation.

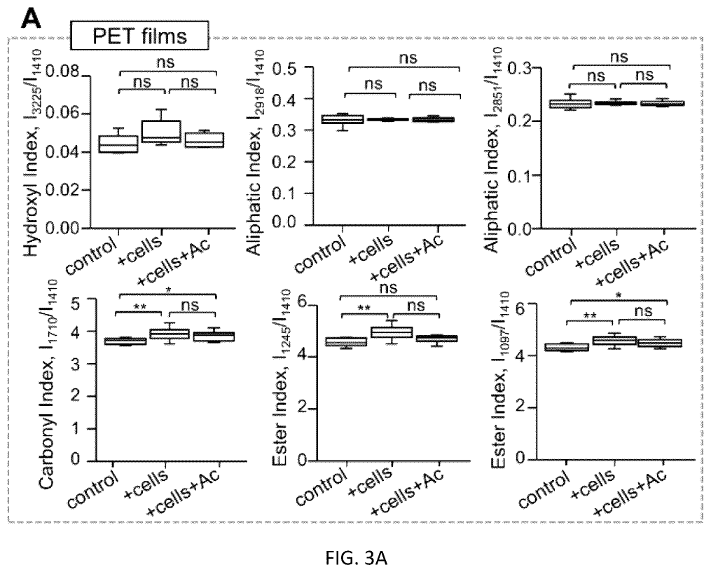

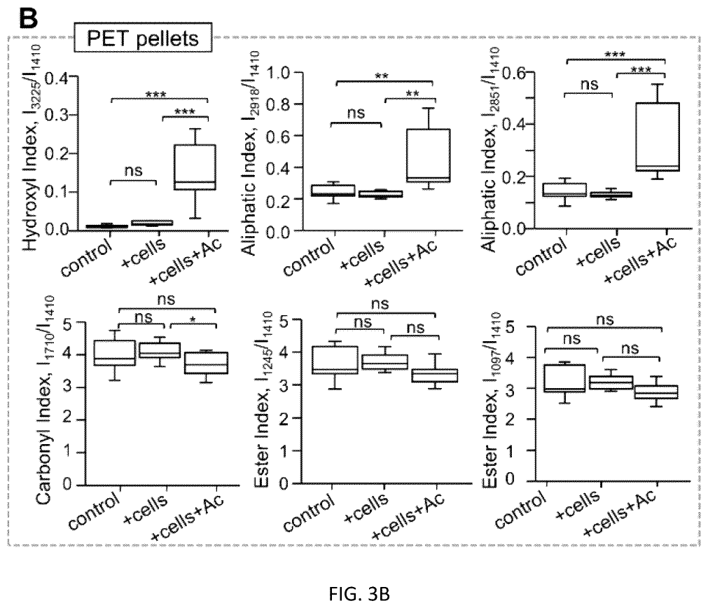

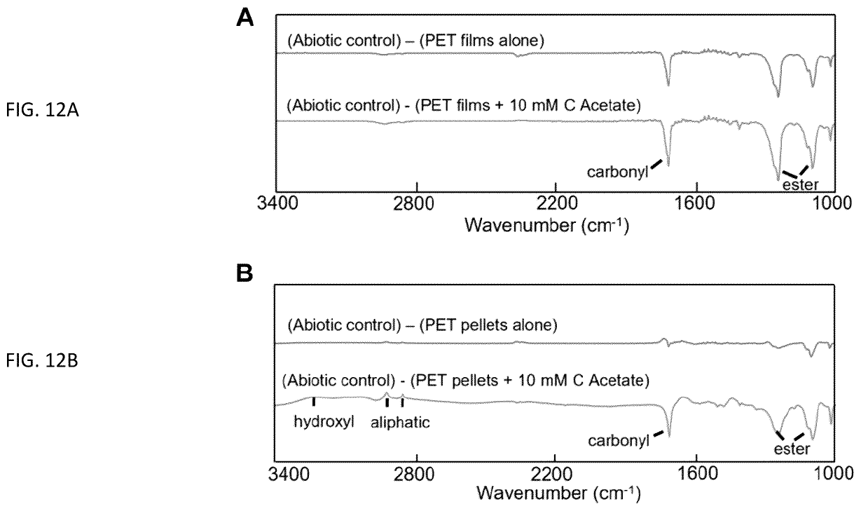

Both oxidative and hydrolytic reactions are proposed to mediate enzymatic degradation of plastics (59, 60). We investigated which reaction mechanism may be responsible for the PET biofragmentation by C. testosteroni KF-1 by monitoring changes in specific functional groups on the surface of the PET films and pellets (FIG. 3A, 3B). We monitored the following functional groups by ATR-FTIR spectroscopy: a broad O—H stretching band at 3263 cm⁻¹ in the hydroxyl group (61), aliphatic C—H stretching vibration bands at 2918 and 2851 cm⁻¹ (62), a stretching band of carbonyl group at 1710 cm⁻¹ (63), and two C—O stretching bands in the ester group at 1245 and 1097 cm-1 (64) (FIGS. 12A and 12B). To compare the change in these characteristic vibrational bands across the different conditions, we normalized the data by the intensity of the CH₂ bending band at 1410 cm⁻¹, as previously described (65, 66). With the PET films, a small increase (5%, P<0.01) in the intensity of carbonyl and ester groups was observed, but no significant change in the intensity of hydroxyl or aliphatic groups (FIG. 3A). The FTIR data with the PET films thus highlighted minor occurrences of oxidative changes on the film surface. Conversely, with the PET pellets, there was pronounced increase in the intensities of both hydroxyl group (13-fold increase, P<0.001) and aliphatic group (69% increase, P<0.01) compared to the abiotic control (FIG. 3B). The significant emergence of hydroxyl signature on the PET pellets implied the substantial fragmentation of these pellets was due to hydrolytic cleavage. In sum, the measurable increase in both the carbonyl and hydroxyl indexes implied occurrences of both oxidation and hydrolysis reactions, respectively, with a clear predominance of hydrolysis reactions (FIG. 3A, 3B).

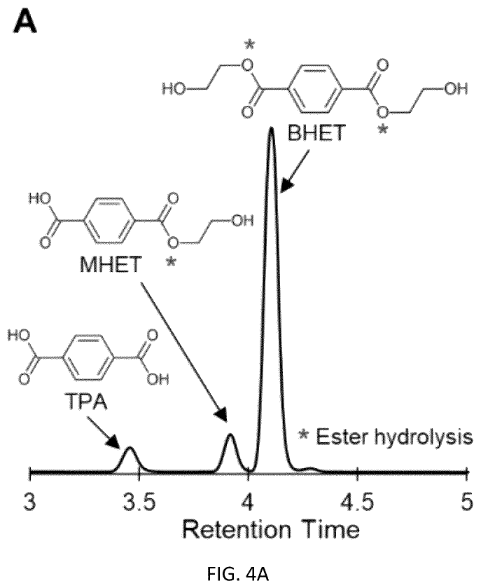

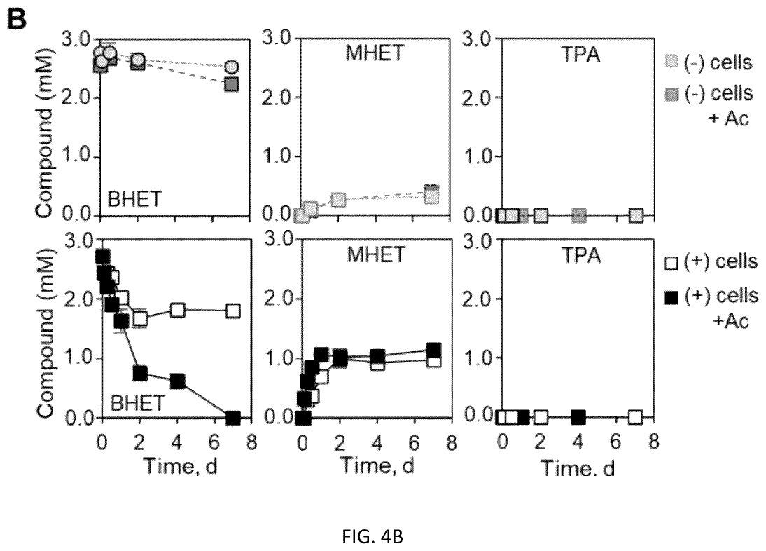

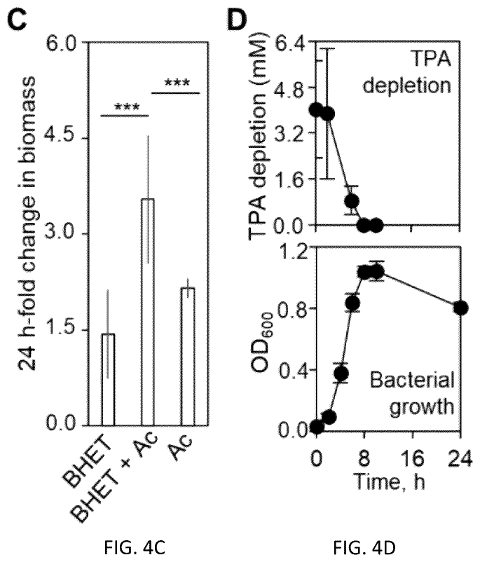

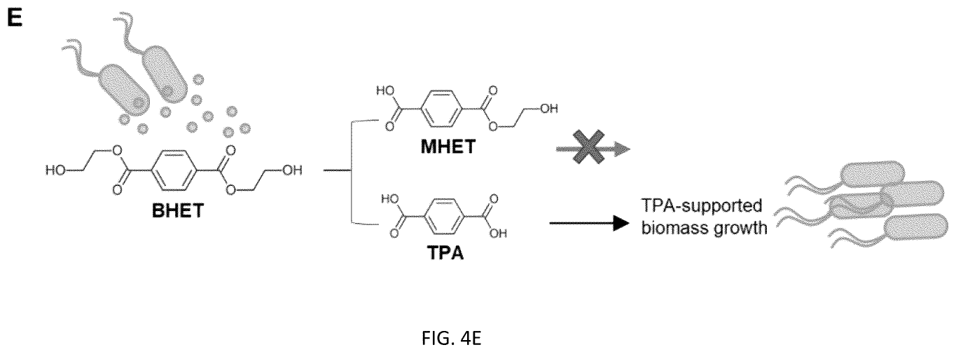

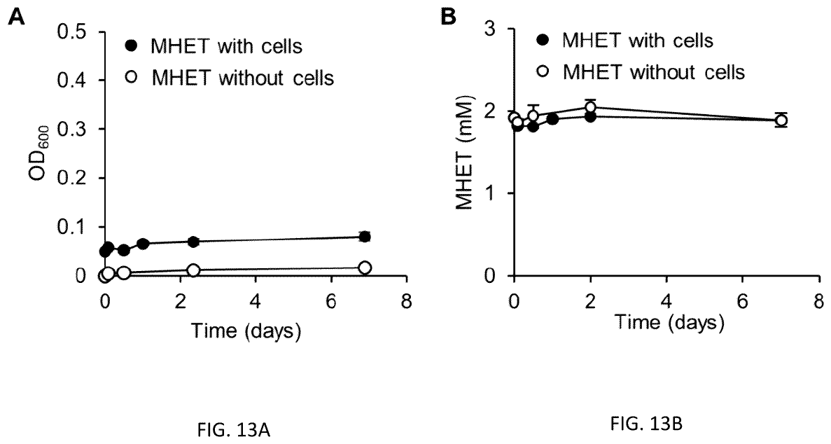

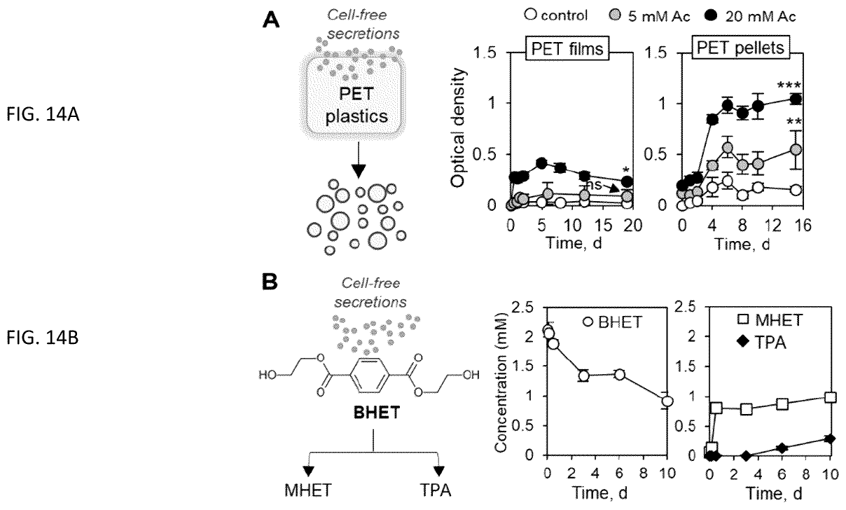

To corroborate further the conclusion from the ATR-FTIR data, UHPLC was used to monitor the hydrolysis of BHET, a PET-oligomer analog, into MHET, a breakdown hydrolytic product of PET, and TPA, a PET monomer from double hydrolysis of BHET (FIG. 4A). During a 7 day incubation of the cells with BHET (2.5 mM) as the sole carbon source, the hydrolytic conversion of 32% of the BHET concentration to MHET was obtained, which stayed constant after reaching a plateau (FIG. 4B). This latter finding along with growth experiments with the C. testosteroni cells led to the conclusion that MHET was unlikely to undergo subsequent hydrolysis to TPA or to support cell growth (FIGS. 13A and 13B). However, direct BHET hydrolysis to TPA via cleavage of the two ester bonds, which has been reported previously with Candida antarctica (67), could not be confirmed here with C. testosteroni due to the absence of measurable TPA; this lack of TPA in solution was attributed to rapid assimilation of this bioavailable carbon source by the cells (44) (FIG. 4B). In fact, a near two-fold or 4.5-fold increase was obtained in cell biomass on BHET alone (P<0.001) or BHET supplemented with acetate (P<0.001), respectively; the cell growth was 37% less on acetate alone than on both BHET and acetate (P<0.001), thus indicating that the hydrolysis of BHET produced bioavailable carbons (FIG. 4C). Moreover, in a separate experiment with TPA as a sole carbon source, the bacterial cells fully depleted TPA and grew rapidly on this PET monomer with a doubling time of 19.3+0.1 min-1 (FIG. 4D); by contrast, ethylene glycol, the other product of BHET hydrolysis, was not able to support the growth of C. testosteroni KF-1 as a sole carbon source (FIGS. 14A and 14B). Therefore, the C. testosteroni KF-1 cells performed double hydrolysis of BHET to generate bioavailable TPA, which can be immediately consumed by the cells in the experiment to support biomass growth.

### Enzymes Involved in the Biofragmentation of PET Plastics.

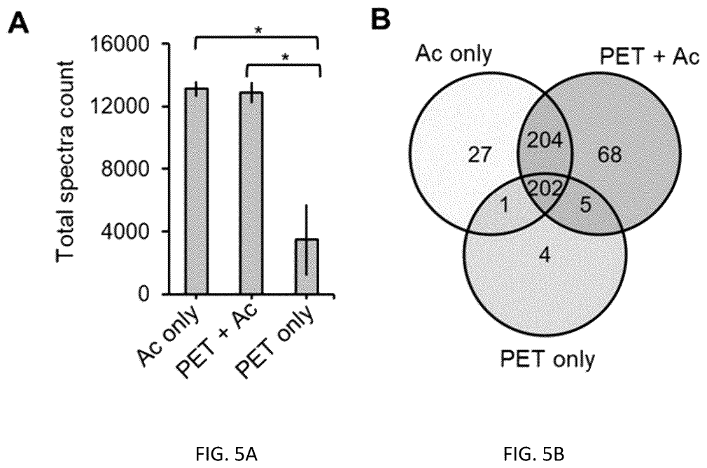

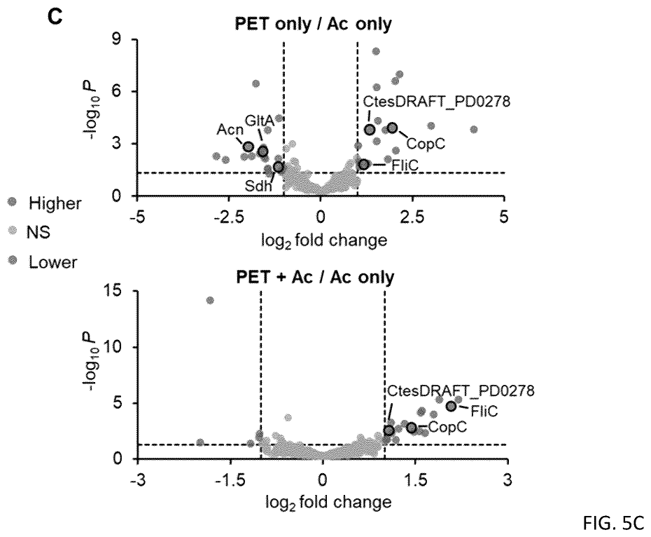

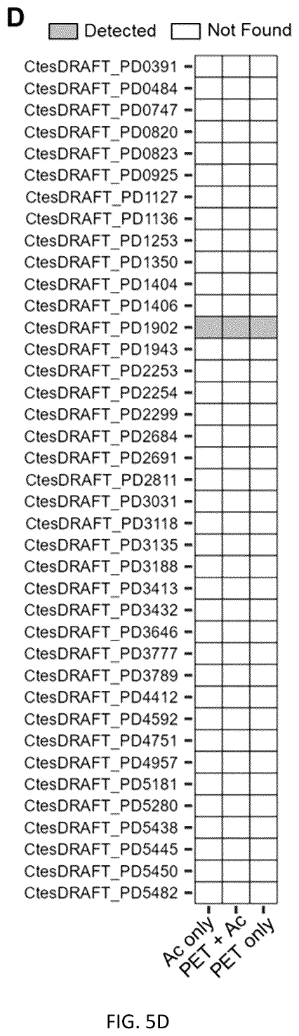

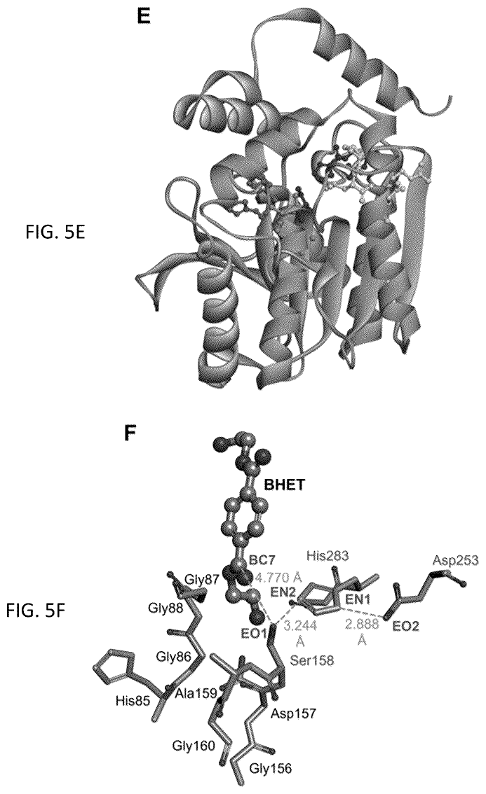

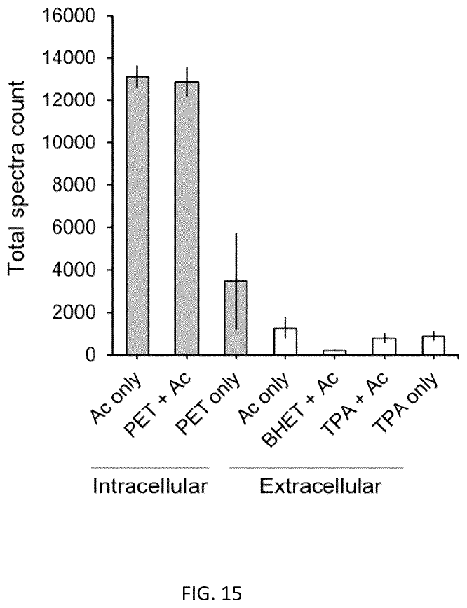

Both extracellular and intracellular proteomics data were obtained in the presence or absence of PET to identify the potential hydrolase(s) generated by C. testosteroni KF-1 that are responsible for PET hydrolysis (FIG. 15). Only the intracellular proteomics datasets were used for comprehensive analysis across the different conditions due to the low coverage of the extracellular proteomics datasets, which represented only 10% of the intracellular ones (P<0.001) (FIG. 15). Compared to growth on PET alone, we obtained up to 4-fold more (P<0.05) identified protein spectra count with cells grown on both PET and acetate or on acetate alone (FIG. 5A). Most proteins identified (>95%) in cells grown on PET alone were also present in the acetate-only condition (FIG. 5B) and we observed some notable relative abundances of select proteins. First, comparing when PET was the sole substrate to the acetate-only condition, there was 2.2-fold to 4-fold lower abundance of enzymes (citrate synthase, aconitate hydratase, and succinate dehydrogenase) involved in the tricarboxylic acid (TCA) cycle (P<0.01) (FIG. 5C). In contrast to the routing of PET-derived aromatic carbons first through a cleavage pathway prior to entry into the TCA cycle, there would be direct influx of acetate-derived carbons into the TCA cycle, thus explaining the need for relatively higher abundance of enzymes in the TCA cycle for the acetate-only condition, especially when regulation for the initial aromatic catabolism has been reported to be via transcriptional regulation of the cleavage pathway (44). Second, comparing again the PET-only condition to the acetate-only condition, we identified 2.1-fold to 4.2-fold higher abundance of a flagellin protein (FliC, P<0.05), a copper resistant protein (CopC, P<0.001) and an organic hydroperoxide resistance protein (CtesDRAFT_PD0278, P<0.01) in the presence of PET (FIG. 5C), implying higher cell mobility and stress, which may facilitate the biodeteriorationactivity observed on the solid plastic surface.

The enzymes that were uniquely found in cells incubated with PET alone or with acetate were associated primarily with ribosomal protein, transcriptional regulators, and flagellin synthesis (Table 1, FIGS. 9A-9C). Interestingly, of the 39 hydrolases annotated in the C. testosteroni KF-1 genome, the only one identified in our proteomics data was CtesDRAFT_PD1902, an esterase/lipase that was present in the cells grown on PET alone, PET with acetate, and acetate alone (FIG. 5C). There was no significant difference in the abundance of the identified esterase when comparing PET alone to acetate alone (P=0.35), or PET with acetate to acetate alone (P=0.25), implying that C. testosteroni constitutively produce this esterase, irrespective of its exposure to PET specifically. Comparisons of the percent identity of CtesDRAFT_PD1902 sequence with 81 known sequences of PET-degrading enzymes (68, 69) revealed that this esterase/lipase was not a homologue (<20% identity) of any known cutinase/lipase, PETase, BHETase or MHETase reported in other microbes (Supplemental dataset 1). Based on sequence similarity, a homology model structure of CtesDRAFT_PD1902 was constructed with Est8, a structurally characterized esterase cloned from a microbial consortium that exhibited diesel oil-degrading capability (70) (FIG. 5D). Interestingly, there were several binding motifs previously found to be conserved in esterases with reported PET hydrolase activity that were also found in our identified esterase (71-76): the residues in the catalytic triad (Ser158. Asp253, and His283), the ⁸⁵HGGG⁸⁸ motif that constitutes the oxyanion hole, and two esterase catalytic motifs (¹⁵⁶GXSXG¹⁶⁰ and ²⁵³DPXXD²⁵⁷) (FIG. 5D).

TABLE 1
Enzymes uniquely identified in PET only condition
relative to acetate only condition

| Locus tag | Function |
|---|---|
| CtesDRAFT_PD0030 | Transcriptional_regulator, _LysR_family |
| CtesDRAFT_PD0333 | TonB dependent receptor |
| CtesDRAFT_PD1318 | TolA_protein |
| CtesDRAFT_PD1422 | SSU_ribosomal_protein_S2p |
| CtesDRAFT_PD2655 | Formate_dehydrogenase_O_beta_subunit |
| CtesDRAFT_PD3077 | DnaJ-class_molecular_chaperone_CbpA |
| CtesDRAFT_PD3217 | DNA-binding_protein_HU-beta |
| CtesDRAFT_PD4753 | LemA_family_protein |
| CtesDRAFT_PD5366 | Urease_alpha_subunit |

TABLE 2
Enzymes uniquely identified in PET with acetate condition relative to acetate only condition

| Locus tag | Function |
|---|---|
| CtesDRAFT_PD0163 | SSU_ribosomal_protein_S13p_(S18e) |
| CtesDRAFT_PD0167 | LSU_ribosomal_protein_L17p |
| CtesDRAFT_PD0184 | 2-hydroxy-3-oxopropionate_reductase_(EC_1.1.1.60) |
| CtesDRAFT_PD0289 | Acetylornithine_deacetylase |
| CtesDRAFT_PD0333 | TonB-dependent_receptor; _Outer_membrane_receptor_for_ferrienterochelin_and_colicins |
| CtesDRAFT_PD0361 | dTDP-glucose_4,6-dehydratase_(EC_4.2.1.46) |
| CtesDRAFT_PD0367 | Putative_virion_core_protein_(lumpy_skin_disease_virus) |
| CtesDRAFT_PD0408 | TRAP-type_C4-dicarboxylate_transport_system, _small_permease_component |
| CtesDRAFT_PD0435 | CzcABC_family_efflux_RND_transporter, _membrane_fusion_protein |
| CtesDRAFT_PD0492 | TldD_protein, _part_of_TldE/TldD_proteolytic_complex |
| CtesDRAFT_PD0530 | Thiol:disulfide_interchange_protein_DsbC |
| CtesDRAFT_PD0550 | Hydroxymethylpyrimidine_phosphate_kinase_ThiD_(EC_2.7.4.7) |
| CtesDRAFT_PD0654 | Putative_transmembrane_protein |
| CtesDRAFT_PD0688 | Zinc_protease |
| CtesDRAFT_PD0738 | Methionyl-tRNA_synthetase_(EC_6.1.1.10) |
| CtesDRAFT_PD0800 | Fumarylacetoacetase_(EC_3.7.1.2) |
| CtesDRAFT_PD0801 | 3-hydroxybutyrate_dehydrogenase_(EC_1.1.1.30) |
| CtesDRAFT_PD0836 | L-carnitine_dehydratase/bile_acid-inducible_protein_F |
| CtesDRAFT_PD0868 | Transaldolase_(EC_2.2.1.2) |
| CtesDRAFT_PD1016 | SSU_ribosomal_protein_S20p |
| CtesDRAFT_PD1034 | Rrf2-linked_NADH-flavin_reductase |
| CtesDRAFT_PD1103 | Aerobic_carbon_monoxide_dehydrogenase_(quinone), _small_chain_(EC_1.2.5.3) |
| CtesDRAFT_PD1362 | Murein_hydrolase_activator_NlpD |
| CtesDRAFT_PD1411 | Chorismate_synthase_(EC_4.2.3.5) |
| CtesDRAFT_PD1415 | Soluble_lytic_murein_transglycosylase_and_related_regulatory_proteins |
| CtesDRAFT_PD1422 | SSU_ribosomal_protein_S2p_(SAe) |
| CtesDRAFT_PD1482 | Phosphoadenylyl-sulfate_reductase_[thioredoxin]_(EC_1.8.4.8) |
| CtesDRAFT_PD1527 | SWIB/MDM2_domain-containing_proteins |
| CtesDRAFT_PD1534 | LSU_ribosomal_protein_L20p |
| CtesDRAFT_PD1620 | Fructose-1,6-bisphosphatase, _type_I_(EC_3.1.3.11) |
| CtesDRAFT_PD1628 | Thiamin-phosphate_pyrophosphorylase_(EC_2.5.1.3) |
| CtesDRAFT_PD1639 | Peptidyl-prolyl_cis-trans_isomerase_(EC_5.2.1.8) |
| CtesDRAFT_PD1782 | Prolidase_(EC_3.4.13.9) |
| CtesDRAFT_PD1871 | UDP-glucose_6-dehydrogenase_(EC_1.1.1.22) |
| CtesDRAFT_PD1908 | Iron-sulfur_cluster_regulator_IscR |
| CtesDRAFT_PD1958 | RND_efflux_system, _inner_membrane_transporter |
| CtesDRAFT_PD2492 | Protein-L-isoaspartate_O-methyltransferase_(EC_2.1.1.77) |
| CtesDRAFT_PD2496 | Phosphoglucomutase_(EC_5.4.2.2)_@_Phosphomannomutase_(EC_5.4.2.8) |
| CtesDRAFT_PD2504 | Predicted_metal-dependent_hydrolase_with_the_TIM-barrel_fold |
| CtesDRAFT_PD2537 | Ribose-5-phosphate_isomerase_A_(EC_5.3.1.6) |
| CtesDRAFT_PD2588 | Succinyl-CoA:3-ketoacid-coenzyme_A_transferase_subunit_B_(EC_2.8.3.5) |
| CtesDRAFT_PD3077 | DnaJ-class_molecular_chaperone_CbpA |
| CtesDRAFT_PD3169 | MoxR-like_ATPases |
| CtesDRAFT_PD3217 | DNA-binding_protein_HU-beta |
| CtesDRAFT_PD3230 | 2-isopropylmalate_synthase_(EC_2.3.3.13) |
| CtesDRAFT_PD3261 | Nucleoid-associated_protein_YaaK |
| CtesDRAFT_PD3322 | Phage_recombination_protein_Bet |
| CtesDRAFT_PD3595 | Amidophosphoribosyltransferase_(EC_2.4.2.14) |
| CtesDRAFT_PD3738 | CoA-transferase_subunit_alpha, _IpdA |
| CtesDRAFT_PD3877 | Deoxyuridine_5′-triphosphate_nucleotidohydrolase_(EC_3.6.1.23) |
| CtesDRAFT_PD4127 | LSU_ribosomal_protein_L28p_@_LSU_ribosomal_protein_L28p, _zinc-independent |
| CtesDRAFT_PD4324 | Ubiquinol-cytochrome_C_reductase_iron-sulfur_subunit_(EC_1.10.2.2) |
| CtesDRAFT_PD4402 | Type_IV_pilus_biogenesis_protein_PilM |
| CtesDRAFT_PD4510 | Lactam_utilization_protein_LamB |
| CtesDRAFT_PD4563 | Excinuclease_ABC_subunit_A, _dimeric_form |
| CtesDRAFT_PD4568 | Acetate_permease_ActP_(cation/acetate_symporter) |
| CtesDRAFT_PD4621 | Chemotaxis_regulator_-_transmits_chemoreceptor_signals_to_flagellar_motor_components_CheY |
| CtesDRAFT_PD4644 | Flagellin_protein_FlaA |
| CtesDRAFT_PD4645 | Flagellar_cap_protein_FliD |
| CtesDRAFT_PD4654 | Flagellar_hook-associated_protein_FlgL |
| CtesDRAFT_PD4678 | Ferric_iron_ABC_transporter, _iron-binding_protein |
| CtesDRAFT_PD4741 | LSU_ribosomal_protein_L11p_(L12e) |
| CtesDRAFT_PD4743 | Protein_translocase_subunit_SecE |
| CtesDRAFT_PD4753 | LemA_family_protein |
| CtesDRAFT_PD4905 | Glycolate_dehydrogenase_(EC_1.1.99.14), _iron-sulfur_subunit_GlcF |
| CtesDRAFT_PD5013 | Pyrimidine_permease |
| CtesDRAFT_PD5112 | N-acetyl-gamma-glutamyl-phosphate_reductase_(EC_1.2.1.38) |
| CtesDRAFT_PD5157 | Protein_translocase_subunit_SecD |
| CtesDRAFT_PD5209 | Barstar, _ribonuclease_(Barnase)_inhibitor |
| CtesDRAFT_PD5249 | PTS_IIA-like_nitrogen-regulatory_protein_PtsN |
| CtesDRAFT_PD5310 | N5-carboxyaminoimidazole_ribonucleotide_synthase_(EC_6.3.4.18) |
| CtesDRAFT_PD5427 | Ribulose-phosphate_3-epimerase_(EC_5.1.3.1) |
| CtesDRAFT_PD5443 | NADPH_dependent_aldo-keto_reductase_YajO |

Importantly, optimized binding of BHET in the predicted binding site of the structural model of our identified esterase revealed key distances between BHET atoms and catalytic residues in the enzyme that were consistent with previous reports from PETase enzymes (FIG. 5E) (70, 76). Specifically, the distance between EO2 of Asp253 and EN2 of His283 was 2.888 Å (compared to 2.633 Å (70)), the distance between EN1 of His283 and EO1 of Ser158 was 3.244 Å (compared to 2.664 Å (70)), and the distance between EO1 of Ser158 and the carbonyl carbon BC7 of BHET was 4.770 Å (FIG. 5E). The latter distance was within 4% of the analogous distance between the catalytic serine and the carbonyl carbon of nP-hexanoate (4.6 Å) in a crystal structure of another 10 alkaline esterase (E53; PDB: 6KEU) (76) which has been compared previously with Est8 and PETase (70, 77). This distance was reported to be catalytically important due to the requirement of the serine residue to perform a nucleophilic attack on the carbonyl carbon of the ester moiety in the bound substrate. Our modeling investigations thus revealed that the esterase CtesDRAFT_PD1902 produced in both the presence and absence of PET by C. testosteroni exhibited the required binding conformation to catalyze the hydrolysis of BHET, a PET oligomer. Therefore, we hypothesize that C. testosteroni produced constitutively PET-degrading enzymes that do not require the presence of PET to trigger their production.

Constitutive Hydrolase Production for the Breakdown of PET Polymer and PET Oligomer by C. testosteroni.

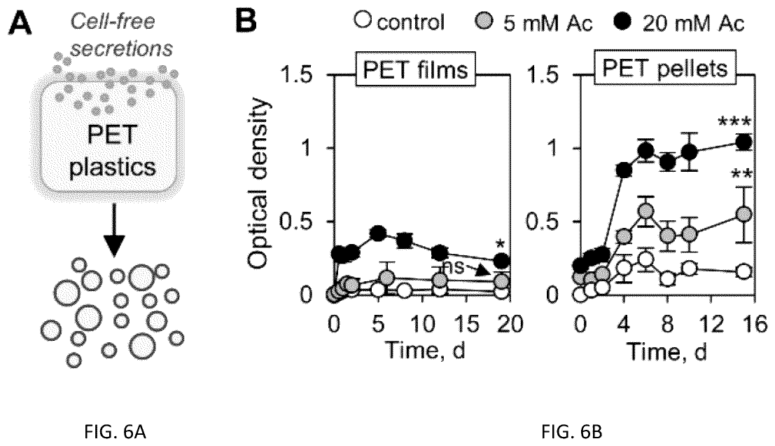

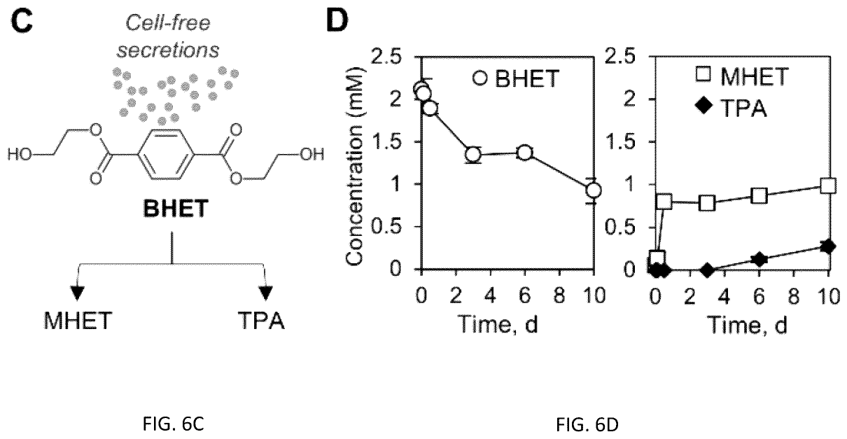

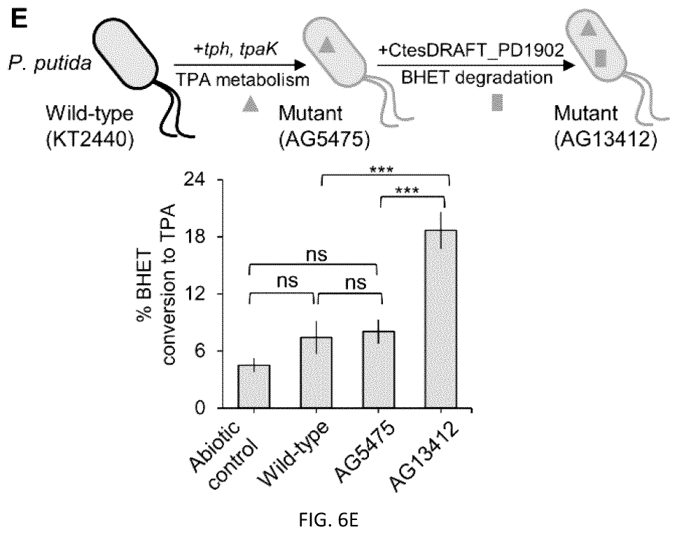

To test this hypothesis, we investigated the fragmentation activity of the enzymes in the secretions of the acetate-fed C. testosteroni KF-1 towards the PET films and pellets (FIG. 6A, 6B). The release of MPs was observed after incubating PET films or pellets with the cell secretions (FIG. 6B), thus indicating that C. testosteroni KF-1 secretes enzymes to catalyze the hydrolysis of PET polymer despite the absence of PET in the growth medium. Furthermore, we incubated cell-free secretions from acetate-grown cells at pH 7 with BHET (2.1 mM) and obtained a 56% depletion in the BHET concentration accompanied by the production and accumulation of both MHET and TPA, the two subsequent breakdown products from single and double hydrolysis reactions of BHET (FIG. 6C, 6D). Notably, the accumulation of MHET and TPA accounted for 44% and 13% of the starting BHET molar concentration, respectively, correlating with the 56% loss of BHET (FIG. 6D). The presence of TPA in the cell-free reaction contrasts with the immediate consumption of TPA when the C. testosteroni cells were present (FIG. 4B), which was consistent with the rapid assimilation of TPA by C. testosteroni.

To confirm that the identified hydrolase (CtesDRAFT_PD1902) is involved in conferring hydrolytic activity, we genetically modified a previously engineered P. putida KT2440 strain (AG5475), which contains a TPA-utilization pathway adopted from Comamonas sp. E6 (52), to express the CtesDRAFT_PD1902 gene identified here from C. testosteroni KF-1, resulting in strain AG13412 (FIG. 6E); the wild-type KT2440 strain does not possess either TPA-utilization or BHET-degrading capabilities. After growing all three P. putida strains (KT2440, AG5475, AG13412) on BHET and acetate as a co-substrate, we did not obtain BHET conversion to TPA by the wild type strain (P=0.16) nor the AG5475 strain (P=0.08) relative to the abiotic control (FIG. 6E). However, endowed with the CtesDRAFT_PD1902 gene, AG13412 was able to achieve nearly 3-fold more of BHET conversion to TPA than the wild-type and AG5475 strains (FIG. 6E) (P<0.001), thus demonstrating that the identified hydrolase encoded by the CtesDRAFT_PD1902 gene in C. testosteroni would facilitate fragmentation and hydrolysis of PET polymers and oligomers.

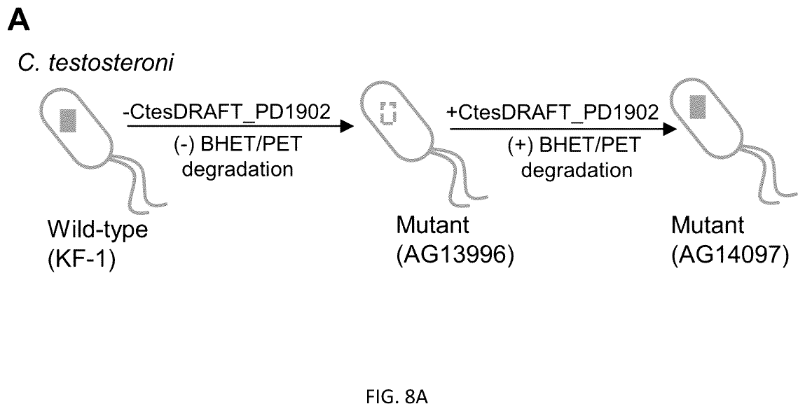

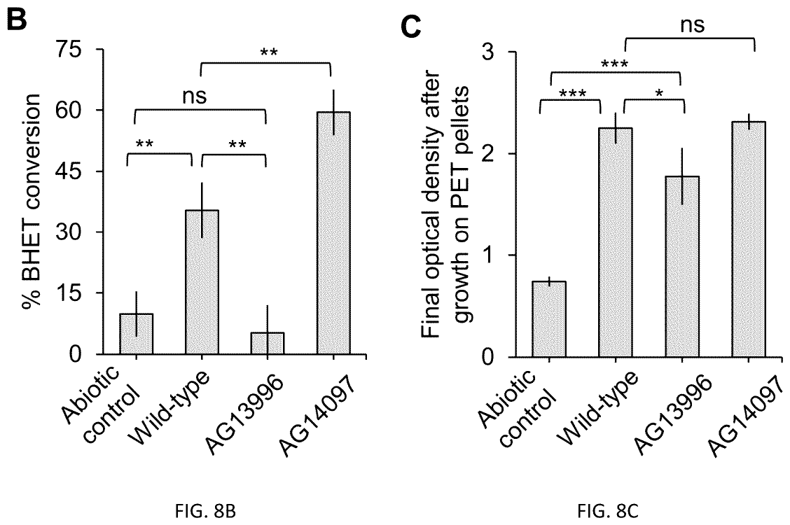

To verify that the identified hydrolase (CtesDRAFT_PD1902) was involved in conferring hydrolytic activity in C. testosteroni KF-1, we constructed a mutant strain (AG1399 6) that lacked this hydrolase gene; we also prepared a strain (AG14097) in which the gene was re-inserted (FIG. 8A). Without CtesDRAFT_PD1902, strain AG13996 lost the ability to degrade the PET oligomer, BHET, thus confirming that the identified hydrolase for the PETase-like activity exhibited by C. testosteroni KF-1 (FIG. 8B). Remarkably, following re-insertion of the hydrolase gene with a Ptac promoter in the deletion mutant, the resulting strain AG14097 recovered the function of BHET degradation, at 69% higher than the wild-type C. testosteroni KF-1 (FIG. 8B). Additionally, we tested the possibility of both mutant strains to degrade PET pellets. The final optical density with strain AG13996 was 21% lower compared to wild-type C. testosteroni strain (P<0.05), but still 2.4-fold higher than the abiotic control (P<0.001) (FIG. 8C). With strain AG14097 with the re-inserted gene, the final optical density was not significantly different from the wild-type culture (FIG. 8C). These data thus indicated that the identified hydrolase contributed to a significant extent to the biofragmentation of PET polymer, but was involved likely in concert with other enzymes because the ability to degrade the PET polymer was not lost completely when the gene was removed (FIG. 8C). For instance, in the extracelluar proteomics data obtained with secretions from C. testosteroni KF-1 grown on acetate only, we had identified CtesDRAFT_PD3135, which was previously characterized as a steroid esterase involved in a steroid cleavage pathway. It is possible this additional hydrolase (CtesDRAFT_PD3135) identified in the Comomonas secretions could also participate along with CtesDRAFT_PD1902 to enhance PET breakdown, but this remains to be confirmed.

### Discussion

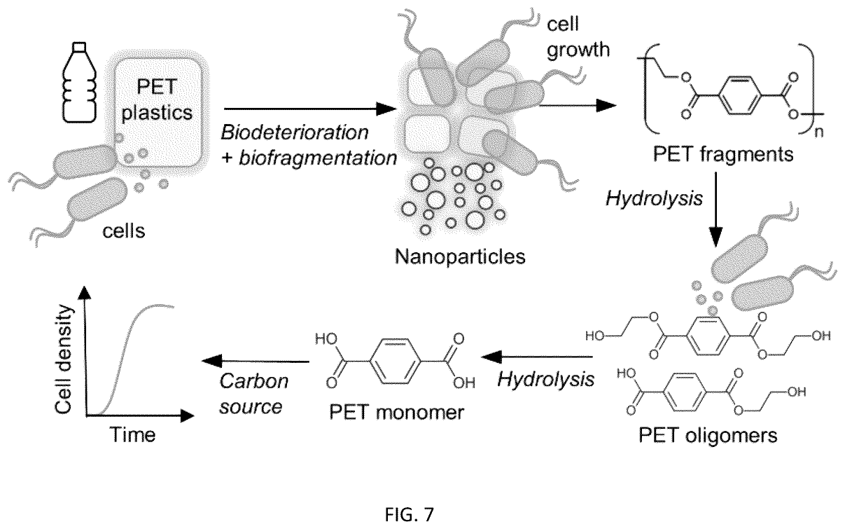

Wastewater treatment plants represent an important repository of MPs materials, including notably PET. Wastewater-residing bacteria with plastics-degrading capabilities play an important role in the fate of these materials. Here we combined microscopic and spectroscopic techniques with proteomics, structural modeling, and genetics to investigate the mechanisms that facilitate reported enrichment of Comamonas-related species on PET microplastics in WWTPs and rivers (33-35). In sum, we demonstrate that C. testosteroni produces secretions containing hydrolases that can facilitate fragmentation of PET plastics to generate NPs and breakdown products available for bacterial assimilation (FIG. 7). Moreover, despite similar surface chemistry, we found that the rough-like surface of the PET pellets was more amendable to biofragmentation than the smooth-like surface of the PET films, indicating a dependence of biofragmentation on the plastics morphology. This finding led us to posit that PET materials with an amorphous-like morphology would be more amenable to fragmentation by wastewater Comamonas than those with crystalline-like morphology, as proposed previously (78, 79).

Several steps are documented systematically in the bioconversion of PET by C. testosteroni KF-418 1, including biodeterioration and biofragmentation to promote the generation of enzyme binding sites with the release of MPs and NPs, the hydrolysis of PET oligomer, and the assimilation of PET monomer that supports bacterial growth (FIG. 7). The data suggest the possible in situ production of PET NPs in WWTPs by wastewater Comamonas, which can potentially be harmful to biota in receiving waters (12, 13). However, evidence of complete degradation of the PET materials to support bacterial growth implies that C. testosteroni cells exhibit attractive traits that can be exploited for engineered bioconversion of plastics. Based on bioinformatics query of the genome, it was previously concluded that Comamonas sp E6 do not possess homologs of known PETases (51), similar to our conclusion here with C. testosteroni KF-1. Importantly, we identified a hydrolase in C. testosteroni KF-1 with confirmed catalytic activity for the hydrolysis of a PET oligomer. Notably, the expression of this hydrolase in C. testosteroni KF-1 was observed during growth on either only PET or only acetate, a common volatile fatty acid found in WWTP solutions (54-57), as the sole carbon source. It is well known that C. testosteroni and related bacteria have a preference for gluconeogenic organic substrates, which include short-chain fatty acids such as acetate and succinate (80, 81). Therefore, we posit that volatile fatty acids widely found in WWTPs can promote secretion of hydrolases to initiate the biotransformation of PET plastics by wastewater Comamonas.

Based on the complex composition of plastic wastes in WWTPs (14, 15), the degradation capability of C. testosteroni and related species towards other plastics materials are worthy of consideration. For instance, C. testosteroni has been shown to grow on dicarboxylates of various carbon lengths (from six carbons to ten carbons) (82), all of which are potential breakdown products of polypropylene and polyethylene plastics (83, 84). Whether the multiple hydrolases in C. testosteroni and other wastewater Comamonas spp. are capable of degrading different plastics polymers have not yet been explored. Given the diversity of microbial communities in WWTPs (85), future investigations also need to consider the effect of interspecies interactions alongside Comamonas on the fate of PET and other plastics in wastewaters.

Finally, it is worthwhile to note that C. testosteroni produces polyhydroxyalkanoate, a polymer widely considered an important precursor to biodegradable plastics (86-89). Therefore, in addition to the depolymerization and assimilation steps, further conversion of the PET-derived carbons to value-added products could be achieved in C. testosteroni as a microbial platform. To leverage this microbial platform toward plastic upcycling, future research needs to evaluate and optimize the channeling of the PET-derived compounds to high yields of polyhydroxyalkanoate or other value-added products.

### Summary of Materials and Methods

Details for materials and methods are provided in SI Appendix, which include the bacterial cultivation, tracking of PET-related compounds using UHPLC, surface morphology and chemistry characterization, nanoparticle visualization and quantification, proteomics, protein homology modeling and mutant construction and examination. In brief, C. testosteroni KF-1 was cultivated using either PET polymers or oligomers with or without acetate as a co-substrate. Cell growth and the release of MPs were monitored by measuring the optical density at 600 nm. A PET oligomer (i.e., BHET) and its hydrolytic products were determined using UHPLC with Agilent ZORBAX Eclipse Plus C18 column (4.6×100 mm with 5 μm particle size) and an ultraviolet detector at 240 nm. Alterations of the surface morphology and chemistry of PET plastics were analyzed using a Hitachi S-3400 N SEM system and a Bruker Vertex 70 FTIR spectrophotometer, respectively. The release of NPs was visualized and quantified by a JEOL Flash 1400 TEM system and a NanoSight NS300 system, respectively. Samples for intracellular and extracellular proteomics were collected, followed by cell lysis and protein extraction. The protein samples were purified and digested prior to analysis on a Dionex Ultimate 3000 LC system with nanoelectrospray ionization coupled with an Orbitrap Elite mass spectrometer operating in a data-dependent acquisition mode. We used the Discovery Studio modeling package to perform both protein homology modeling for and identified esterase encoded by CtesDRAFT_PD1902 and molecular docking simulation of BHET binding in the predicted substrate binding pocket of the identified esterase. The CtesDRAFT_PD1902 gene from C. testosteroni was codon optimized and cloned into Pseudomonas putida KT2440, a non-PET degrading bacterium, to examine the function of CtesDRAFT_PD1902 in the hydrolysis of the PET oligomer.

### REFERENCES

1. C. M. Rochman, et al., Classify plastic waste as hazardous. Nature 494, 169-171 (2013).
2. K. L. Law, Plastics in the Marine Environment. Annual Review of Marine Science 9, 205-229 (2017).
3. B. Carney Almroth, H. Eggert, Marine Plastic Pollution: Sources, Impacts, and Policy Issues. Review of Environmental Economics and Policy 13, 317-326 (2019).
4. T. van Emmerik, A. Schwarz, Plastic debris in rivers. WIREs Water 7, e1398 (2020).
5. R. N. Cable, et al., Distribution and Modeled Transport of Plastic Pollution in the Great Lakes, the World's Largest Freshwater Resource. Frontiers in Environmental Science 5 (2017).
6. P. L. Corcoran, et al., Hidden plastics of Lake Ontario, Canada and their potential preservation in the sediment record. Environmental Pollution 204, 17-25 (2015).
7. C. Martin, et al., Exponential increase of plastic burial in mangrove sediments as a major plastic sink. Science Advances 6, eaaz5593 (2020).
8. Y. Huo, F. A. Dijkstra, M. Possell, B. Singh, “Chapter One-Plastics in soil environments: All things considered” in Advances in Agronomy, D. L. Sparks, Ed. (Academic Press, 2022), pp. 1-132.
9. Y. Qi, et al., Macro- and micro-plastics in soil-plant system: Effects of plastic mulch film residues on wheat (Triticum aestivum) growth. Science of The Total Environment 645, 1048-1056 (2018).
10. W. Courtene-Jones, N. J. Clark, A. C. Fischer, N. S. Smith, R. C. Thompson, “Ingestion of Microplastics by Marine Animals” in Plastics and the Ocean, (John Wiley & Sons, Ltd, 2022), pp. 349-366.
11. C. Larue, G. Sarret, H. Castillo-Michel, A. E. Pradas del Real, A Critical Review on the Impacts of Nanoplastics and Microplastics on Aquatic and Terrestrial Photosynthetic Organisms. Small 17, 2005834 (2021).
12. J. Saavedra, S. Stoll, V. I. Slaveykova, Influence of nanoplastic surface charge on eco-corona formation, aggregation and toxicity to freshwater zooplankton. Environmental Pollution 252, 715-722 (2019).
13. L. Wang, et al., Environmental fate, toxicity and risk management strategies of nanoplastics in the environment: Current status and future perspectives. Journal of Hazardous Materials 401, 123415 (2021).
14. S. Wolff, J. Kerpen, J. Prediger, L. Barkmann, L. Müller, Determination of the microplastics emission in the effluent of a municipal waste water treatment plant using Raman microspectroscopy. Water Research X 2, 100014 (2019).
15. M. Van Do, T. X. T. Le, N. D. Vu, T. T. Dang, Distribution and occurrence of microplastics in wastewater treatment plants. Environmental Technology & Innovation 26, 102286 (2022).
16. K. Conley, A. Clum, J. Deepe, H. Lane, B. Beckingham, Wastewater treatment plants as a source of microplastics to an urban estuary: Removal efficiencies and loading per capita over one year. Water Research X 3, 100030 (2019).
17. M. Kazour, et al., Sources of microplastics pollution in the marine environment: Importance of wastewater treatment plant and coastal landfill. Marine Pollution Bulletin 146, 608-618 (2019).
18. E. D. Okoffo, S. O'Brien, J. W. O'Brien, B. J. Tscharke, K. V. Thomas, Wastewater treatment plants as a source of plastics in the environment: a review of occurrence, methods for identification, quantification and fate. Environmental Science: Water Research & Technology 5, 1908-1931 (2019).
19. D. Sol, A. Laca, A. Laca, M. Díaz, Approaching the environmental problem of microplastics: Importance of WWTP treatments. Science of The Total Environment 740, 140016 (2020).
20. S. Frehland, R. Kaegi, R. Hufenus, D. M. Mitrano, Long-term assessment of nanoplastic particle and microplastic fiber flux through a pilot wastewater treatment plant using metal-doped plastics. Water Research 182, 115860 (2020).
21. P. Benyathiar, P. Kumar, G. Carpenter, J. Brace, D. K. Mishra, Polyethylene Terephthalate (PET) Bottle-to-Bottle Recycling for the Beverage Industry: A Review. Polymers 14 (2022).
22. M. U. Sheth, et al., Bioengineering a Future Free of Marine Plastic Waste. Frontiers in Marine Science 6 531 (2019).
23. S. Maity, et al., Functional interplay between plastic polymers and microbes: a comprehensive review. Biodegradation 32, 487-510 (2021).
24. X. Qi, et al., Evaluation of PET Degradation Using Artificial Microbial Consortia. Frontiers in Microbiology 12 (2021). 536
25. I. E. Meyer-Cifuentes, et al., Synergistic biodegradation of aromatic-aliphatic copolyester plastic by a marine microbial consortium. Nat Commun 11, 5790 (2020).
26. Z. Cao, W. Yan, M. Ding, Y. Yuan, Construction of microbial consortia for microbial degradation of complex compounds. Frontiers in Bioengineering and Biotechnology 10 (2022).
27. B. Maheswaran, et al., In vivo degradation of polyethylene terephthalate using microbial isolates from plastic polluted environment. Chemosphere 310, 136757 (2023).
28. C. Roberts, et al., Environmental Consortium Containing Pseudomonas and Bacillus Species 543 Synergistically Degrades Polyethylene Terephthalate Plastic. mSphere 5, e01151-20 (2020).
29. T. C. H. Dang, et al., Plastic degradation by thermophilic Bacillus sp. BCBT21 isolated from composting agricultural residual in Vietnam. Adv. Nat. Sci: Nanosci. Nanotechnol. 9, 015014 (2018).
30. S. Tanasupawat, T. Takehana, S. Yoshida, K. Hiraga, K. Oda, Ideonella sakaiensis sp. nov., isolated from a microbial consortium that degrades poly (ethylene terephthalate). International Journal of Systematic and Evolutionary Microbiology 66, 2813-2818 (2016).
31. S. Weigert, et al., Investigation of the halophilic PET hydrolase PET6 from Vibrio gazogenes. Protein Science 31, e4500 (2022).
32. R. Sarkhel, S. Sengupta, P. Das, A. Bhowal, Comparative biodegradation study of polymer from plastic bottle waste using novel isolated bacteria and fungi from marine source. J Polym Res 27, 16 (2019).
33. A. McCormick, T. J. Hoellein, S. A. Mason, J. Schluep, J. J. Kelly, Microplastic is an Abundant and Distinct Microbial Habitat in an Urban River. Environ. Sci. Technol. 48, 11863-11871 (2014).
34. A. R. McCormick, et al., Microplastic in surface waters of urban rivers: concentration, sources, and associated bacterial assemblages. Ecosphere 7, e01556 (2016).
35. S. Martínez-Campos, et al., Characterization of microbial colonization and diversity in reverse osmosis membrane autopsy. dwt 131, 9-29 (2018).
36. S. Martínez-Campos, M. González-Pleiter, F. Fernández-Piñas, R. Rosal, F. Leganés, Early and differential 560 bacterial colonization on microplastics deployed into the effluents of wastewater treatment plants. 561 Science of The Total Environment 757, 143832 (2021).
37. Y. Huang, X. Hou, S. Liu, J. Ni, Correspondence analysis of bio-refractory compounds degradation and microbiological community distribution in anaerobic filter for coking wastewater treatment. Chemical Engineering Journal 304, 864-872 (2016).
38. J. Zhan, Y. Han, S. Xu, X. Wang, X. Guo, Succession and change of potential pathogens in the co-composting of rural sewage sludge and food waste. Waste Management 149, 248-258 (2022).
39. H. H. Locher, T. Leisinger, A. M. Cook, Degradation of p-Toluenesulphonic Acid via Sidechain Oxidation, Desulphonation and meta Ring Cleavage in Pseudomonas (Comamonas) testosteroni T-2. Microbiology 135, 1969-1978 (1989).
40. M. Okai, et al., Comamonas sp. 3ah48 is a dibenz [a, h] anthracene-degrading bacterium that is tolerant to heavy metals. Letters in Applied Microbiology 68, 589-596 (2019).
41. J. R. Dutton, W. A. Venables, C. P. Saint, Comamonas acidovorans UCC61 catabolizes o-phthalate via a 4,5-oxygenation pathway that is encoded on a 70 kbp section of plasmid pOPHI bounded by directly repeated sequences. Microbiology 141, 1673-1682 (1995).
42. M. A. Providenti, J. M. O'Brien, J. Ruff, A. M. Cook, I. B. Lambert, Metabolism of Isovanillate, Vanillate, and Veratrate by Comamonas testosteroni Strain BR6020. Journal of Bacteriology 188, 3862-3869 577 (2006).
43. W. M. Kincannon, et al., Biochemical and structural characterization of an aromatic ring-hydroxylating dioxygenase for terephthalic acid catabolismproc Natl Acad Sci USA 119, e2121426119 (2022).
44. R. A. Wilkes, et al., Complex regulation in a Comamonas platform for diverse aromatic carbon metabolism. Nat Chem Biol 1-12 (2023). doi.org/10.1038/s41589-022-01237-7.
45. M. Weiss, et al., Permanent draft genome sequence of Comamonas testosteroni KF-1. Stand in Genomic Sci 8, 239-254 (2013).
46. G. von Haugwitz, et al., Structural Insights into (Tere) phthalate-Ester Hydrolysis by a Carboxylesterase and Its Role in Promoting PET Depolymerization. ACS Catal. 12, 15259-15270 (2022).
47. H. F. Son, et al., Rational Protein Engineering of Thermo-Stable PETase from Ideonella sakaiensis for Highly Efficient PET Degradation. ACS Catal. 9, 3519-3526 (2019).
48. D. Ribitsch, et al., Fusion of Binding Domains to Thermobifida cellulosilytica Cutinase to Tune Sorption Characteristics and Enhancing PET Hydrolysis. Biomacromolecules 14, 1769-1776 (2013).
49. K. Herzog, R.-J. Müller, W.-D. Deckwer, Mechanism and kinetics of the enzymatic hydrolysis of polyester nanoparticles by lipases. Polymer Degradation and Stability 91, 2486-2498 (2006).
50. R. a. Wilkes, L. Aristilde, Degradation and metabolism of synthetic plastics and associated products by Pseudomonas sp.: capabilities and challenges. Journal of Applied Microbiology 123, 582-593 (2017).
51. B. C. Knott, et al., Characterization and engineering of a two-enzyme system for plastics depolymerization. Proc. Natl. Acad. Sci. U.S.A. 117, 25476-25485 (2020).
52. K. P. Sullivan, et al., Mixed plastics waste valorization through tandem chemical oxidation and biological funneling. Science 378, 207-211 (2022).
53. O. F. Brandenberg, O. T. Schubert, L. Kruglyak, Towards synthetic PETtrophy: Engineering Pseudomonas putida for concurrent polyethylene terephthalate (PET) monomer metabolism and PET hydrolase expression. Microb Cell Fact 21, 119 (2022).
54. A. S. Ucisik, M. Henze, Biological hydrolysis and acidification of sludge under anaerobic conditions: The effect of sludge type and origin on the production and composition of volatile fatty acids. Water Research 42, 3729-3738 (2008).
55. E. Reyhanitash, S. R. A. Kersten, B. Schuur, Recovery of Volatile Fatty Acids from Fermented Wastewater by Adsorption. ACS Sustainable Chem. Eng. 5, 9176-9184 (2017).
56. S. Agnihotri, et al., A Glimpse of the World of Volatile Fatty Acids Production and Application: A review. Bioengineered 13, 1249-1275.
57. S. Longo, et al., Recovery of volatile fatty acids from fermentation of sewage sludge in municipal wastewater treatment plants. Bioresource Technology 175, 436-444 (2015).
58. J. Gong, et al., Biodegradation of Microplastic Derived from Poly (ethylene terephthalate) with Bacterial Whole-Cell Biocatalysts. Polymers (Basel) 10, 1326 (2018).
59. N. Mohanan, Z. Montazer, P. K. Sharma, D. B. Levin, Microbial and Enzymatic Degradation of Synthetic Plastics. Frontiers in Microbiology 11 (2020).
60. Z. Cai, et al., Biological Degradation of Plastics and Microplastics: A Recent Perspective on Associated Mechanisms and Influencing Factors. Microorganisms 11, 1661 (2023).
61. S. A. Parry, A. R. Pawley, R. L. Jones, S. M. Clark, An infrared spectroscopic study of the OH stretching frequencies of talc and 10-A phase to 10 GPa. American Mineralogist 92, 525-531 (2007).
62. J. M. Cervantes-Uc, et al., TGA/FTIR studies of segmented aliphatic polyurethanes and their nanocomposites prepared with commercial montmorillonites. Polymer Degradation and Stability 94, 1666-1677 (2009).
63. J. Almond, P. Sugumaar, M. N. Wenzel, G. Hill, C. Wallis, Determination of the carbonyl index of polyethylene and polypropylene using specified area under band methodology with ATR-FTIR spectroscopy. e-Polymers 20, 369-381 (2020).
64. J. Van Der Weerd, A. Van Loon, J. J. Boon, FTIR Studies of the Effects of Pigments on the Aging of Oil. Studies in Conservation 50, 3-22 (2005).
65. M. Drobota, et al., Chemical modification and characterization of poly (ethylene terephthalate) surfaces for collagen immobilization. Open Chemistry 11, 1786-1798 (2013).
66. I. Donelli, G. Freddi, V. A. Nierstrasz, P. Taddei, Surface structure and properties of poly-(ethylene terephthalate) hydrolyzed by alkali and cutinase. Polymer Degradation and Stability 95, 1542-1550 (2010).
67. K. Świderek, et al., Mechanistic studies of a lipase unveil effect of pH on hydrolysis products of small PET modules. Nat Commun 14, 3556 (2023).
68. V. Gambarini, et al., Phylogenetic Distribution of Plastic-Degrading Microorganisms. mSystems 6, 10.1128/msystems.01112-20 (2021).
69. V. Gambarini, et al., PlasticDB: a database of microorganisms and proteins linked to plastic biodegradation. Database (Oxford) 2022, baac008 (2022).
70. M. R. Pereira, et al., From a metagenomic source to a high-resolution structure of a novel alkaline esterase. Appl Microbiol Biotechnol 101, 4935-4949 (2017).
71. H. P. Austin, et al., Characterization and engineering of a plastic-degrading aromatic polyesterase. Proceedings of the National Academy of Sciences 115, E4350-E4357 (2018).
72. B. Liu, et al., Protein Crystallography and Site-Direct Mutagenesis Analysis of the Poly (ethylene 642 terephthalate) Hydrolase PETase from Ideonella sakaiensis. ChemBioChem 19, 1471-1475 (2018).
73. S. Joo, et al., Structural insight into molecular mechanism of poly (ethylene terephthalate) degradation. Nat Commun 9, 382 (2018).
74. T. Fecker, et al., Active Site Flexibility as a Hallmark for Efficient PET Degradation by I. sakaiensis PETase. Biophysical Journal 114, 1302-1312 (2018).
75. X. Han, et al., Structural insight into catalytic mechanism of PET hydrolase. Nat Commun 8, 2106 (2017).
76. Y. Ding, et al., Mechanism and Structural Insights Into a Novel Esterase, E53, Isolated From Erythrobacter longus. Frontiers in Microbiology 12 (2022).
77. C. Jerves, R. P. P. Neves, M. J. Ramos, S. da Silva, P. A. Fernandes, Reaction Mechanism of the PET Degrading Enzyme PETase Studied with DFT/MM Molecular Dynamics Simulations. ACS Catal. 11, 11626-11638 (2021).
78. Y.-H. V. Soong, M. J. Sobkowicz, D. Xie, Recent Advances in Biological Recycling of Polyethylene Terephthalate (PET) Plastic Wastes. Bioengineering 9, 98 (2022).
79. F. Julienne, F. Lagarde, N. Delorme, Influence of the crystalline structure on the fragmentation of weathered polyolefines. Polymer Degradation and Stability 170, 109012 (2019).
80. A. Basu, S. K. Apte, P. S. Phale, Preferential Utilization of Aromatic Compounds over Glucose by Pseudomonas putida CSV86. Applied and Environmental Microbiology 72, 2226-2230 (2006).
81. R. A. Wilkes, J. Waldbauer, L. Aristilde, Analogous Metabolic Decoupling in Pseudomonas putida and Comamonas testosteroni Implies Energetic Bypass to Facilitate Gluconeogenic Growth. mBio 12, e03259-21 (2021).
82. J. Tamaoka, D.-M. Ha, K. Komagata, Reclassification of Pseudomonas acidovorans den Dooren de Jong 1926 and Pseudomonas testosteroni Marcus and Talalay 1956 as Comamonas acidovorans comb. nov. and Comamonas testosteroni comb. nov., with an Emended Description of the Genus Comamonas. International Journal of Systematic and Evolutionary Microbiology 37, 52-59 (1987).
83. S. Karlsson, M. Hakkarainen, A.-C. Albertsson, Dicarboxylic Acids and Ketoacids Formed in Degradable Polyethylenes by Zip Depolymerization through a Cyclic Transition State. Macromolecules 30, 7721-7728 (1997).
84. A. C. Fernandes, Reductive depolymerization as an efficient methodology for the conversion of plastic waste into value-added compounds. Green Chemistry 23, 7330-7360 (2021).
85. D. R. Johnson, T. K. Lee, J. Park, K. Fenner, D. E. Helbling, The functional and taxonomic richness of wastewater treatment plant microbial communities are associated with each other and with ambient nitrogen and carbon availability. Environmental Microbiology 17, 4851-4860 (2015).
86. Roohi, M. R. Zaheer, M. Kuddus, PHB (poly-β-hydroxybutyrate) and its enzymatic degradation. Polymers for Advanced Technologies 29, 30-40 (2018).
87. S. M. Emadian, T. T. Onay, B. Demirel, Biodegradation of bioplastics in natural environments. Waste Management 59, 526-536 (2017).
88. R. Plavec, et al., Recycling possibilities of bioplastics based on PLA/PHB blends. Polymer Testing 92, 679 106880 (2020).
89. E. L. Eronen-Rasimus, P. P. Näkki, H. P. Kaartokallio, Degradation Rates and Bacterial Community Compositions Vary among Commonly Used Bioplastic Materials in a Brackish Marine Environment.Environ. Sci. Technol. 56, 15760-15769 (2022).

Informal Sequence Listing

SEQ ID NO: 1-PD1902 amino acid sequence
maldphlagv lqqlaaanrk staegtpeeg ragylaltrg sltpeqivpv asvqdttvpg gagpvaariy rpegagpfpt
vayfhgggyv ignldthdni creicrgaqa vvvsvdyrla pehpfpagie davaaarwvv anahalggsa tvavagdsag
gnfcavvtqq lrdagialaa qfliypavdh aaaeyasaeq nakgyfleae tmawfynhya gtfpdaldpr laplqaksla
nlpsavivna efdplrdqga ayaealraag gqaeliegag mihgffdmgr wspgaqavit hsierfaall ttrar
SEQ ID NO: 2-Expression plasmid for PD1902

| | |
|---|---|
| 1 | ccaatgatac tgatttttaa ggcgactgat gagtcgcctt ttttttgtct aagaattcat |
| 61 | cagaagaact cgtcaagaag gcgatagaag gcgatgcgct gcgaatcggg agcggcgata |
| 121 | ccgtaaagca cgaggaagcg gtcagcccat tcgccgccaa gctcttcagc aatatcacgg |
| 181 | gtagccaacg ctatgtcctg atagcggtcc gccacaccca gccggccaca gtcgatgaat |
| 241 | ccagaaaagc ggccattttc caccatgata ttcggcaagc aggcatcgcc atgggtcacg |
| 301 | acgagatcct cgccgtcggg catccgcgcc ttgagcctgg cgaacagttc ggctggcgcg |
| 361 | agcccctgat gctcttcgtc cagatcatcc tgatcgacaa gaccggcttc catccgagta |
| 421 | cgtgctcgct cgatgcgatg tttcgcttgg tggtcgaatg ggcaggtagc cggatcaagc |
| 481 | gtatgcagcc gccgcattgc atcagccatg atggatactt tctcggcagg agcaaggtga |
| 541 | gatgacagga gatcctgccc cggcacttcg cccaatagca gccagtccct tcccgcttca |
| 601 | gtgacaacgt cgagcacagc tgcgcaagga acgcccgtcg tggccagcca cgatagccgc |
| 661 | gctgcctcgt cttggagttc attcagggca ccggacaggt cggtcttgac aaaaagaacc |
| 721 | gggcgcccct gcgctgacag ccggaacacg gcggcatcag agcagccgat tgtctgttgt |
| 781 | gcccagtcat agccgaatag cctctccacc caagcggccg gagaacctgc gtgcaatcca |
| 841 | tcttgttcaa tcatgcgaaa cgatcctcat cctgtctctt gatcagatct tgatcccctg |
| 901 | cgccatcaga tccttggcgg caagaaagcc atccagttta ctttgcaggg cttcccaacc |
| 961 | ttaccagagg gcgccccagc tggcaattcc ggttcgcttg ctgtccataa aaccgcccag |
| 1021 | tctagctatc gccatgtaag cccactgcaa gctacctgct ttctctttgc gcttgcgttt |
| 1081 | tcccttgtcc agatagccca gtagctgaca ttcatccggg acgtcgtgcc ccaactgggg |
| 1141 | taacctttga gttctctcag ttgggggatc gatagtcaaa agcctccggt cggaggcttt |
| 1201 | tgactagcac ctcggtacca aattccagaa aagaggcctc ccgaaagggg ggcctttttt |
| 1261 | cgttttggtc cggatccgat atcagtctct atggaggtca ggtatgatta ctattgacaa |
| 1321 | ttaatcatcg gctcgtataa tgtgatcaga cctggaattg tgagcggata acaattgttc |
| 1381 | tacgggtaag ggggtttttt tatggcgctg gatccacatc tagccggcgt tctccagcaa |
| 1441 | cttgcggctg cgaaccgcaa gtcaaccgct gaggggaccc ctgaggaagg ccgagcgggg |
| 1501 | tatctcgcct taacgagagg gagcttaacg ccagaacaga tcgttccagt cgcgagtgta |
| 1561 | caggatacaa ccgtaccggg tggagccggc cctgtggcag cgcgcatcta tagacctgaa |
| 1621 | ggggcgggac cgtttcctac agtggcgtac ttccatggcg gtgggtacgt catcggtaat |
| 1681 | ttggatacac acgacaatat ctgccgtgaa atttgtcgcg gtgcccaagc tgttgtggtt |
| 1741 | agtgtggact atcggttagc cccggaacat cccttcccag ccggaattga agatgcagta |
| 1801 | gcggcagctc ggtgggtcgt cgcgaacgct cacgcgttgg gcggatcagc cactgtagca |
| 1861 | gtggcagggg acagcgcagg cgggaatttc tgtgcggtgg taacccagca gttgcgcgac |
| 1921 | gcaggcatag ctctagctgc gcagttcttg atttaccccg cggttgacca cgcggcggcg |
| 1981 | gagtacgctt cagcagaaca aaatgccaaa ggctattttc tggaggcaga aacgatggct |
| 2041 | tggttctaca accattacgc aggcaccttc cctgacgccc ttgatccccg cttggctccc |
| 2101 | ttacaagcaa agagtctggc taacttgccg tccgctgtga ttgttaacgc cgaattcgac |
| 2161 | ccactaagag atcaaggtgc cgcttacgcc gaggcattaa gagccgctgg tgggcaagca |
| 2221 | gagttgatcg agggcgctgg catgatccac gggttctttg atatggggag atggtcgccg |
| 2281 | ggagcgcagg cggttataac acatagtata gaacgttttg ccgccctgtt gacaactagg |
| 2341 | gctcgataat ctagactcga ggacgaacaa taaggcctcc ctaacggggg gcctttttta |
| 2401 | ttgataacaa aaatccacaa ggaaaaatta aaggggagat aaaatccccc ctttttggtt |
| 2461 | aactgcggcc gcgtcgtggt ttgtctggtc aaccaccgcg gtctcagtgg tgtacggtac |
| 2521 | aaaccccgac gctagctgcg ggtgccaggg cgtgcccttg ggctccccgg gcgcgtactc |
| 2581 | catcggaaag aacatgtgag caaaaggcca gcaaaaggcc aggaaccgta aaaaggccgc |
| 2641 | gttgctggcg tttttccata ggctccgccc ccctgacgag catcacaaaa atcgacgctc |
| 2701 | aagtcagagg tggcgaaacc cgacaggact ataaagatac caggcgtttc cccctggaag |
| 2761 | ctccctcgtg cgctctcctg ttccgaccct gccgcttacc ggatacctgt ccgcctttct |
| 2821 | cccttcggga agcgtggcgc tttctcatag ctcacgctgt aggtatctca gttcggtgta |
| 2881 | ggtcgttcgc tccaagctgg gctgtgtgca cgaacccccc gttcagcccg accgctgcgc |
| 2941 | cttatccggt aactatcgtc ttgagtccaa cccggtaaga cacgacttat cgccactggc |
| 3001 | agcagccact ggtaacagga ttagcagagc gaggtatgta ggcggtgcta cagagttctt |
| 3061 | gaagtggtgg cctaactacg gctacactag aagaacagta tttggtatct gcgctctgct |
| 3121 | gaagccagtt accttcggaa aaagagttgg tagctcttga tccggcaaac aaaccaccgc |
| 3181 | tggtagcggt ggtttttttg tttgcaagca gcagattacg cgcagaaaaa aaggatctca |
| 3241 | agaagatcct ttgatctttt ctacggggtc tgacgctcag tggaacgaaa actcacgtta |
| 3301 | agggattttg gtcatgagat tatcaaaaag gatcttcacc tagatccttt taaattaaaa |
| 3361 | atgaagtttt aaatcaatct aaagtatata tgagtaaact tggtctgaca gttaccaatg |
| 3421 | cttaatcagt gaggcaccta tctcagcgat ctgtctattt cgttcatcca tagttgcctg |
| 3481 | actccccgtc gtgtagataa ctacgatacg ggagggctta ccatctggcc ccagtgctgc |
| 3541 | aatgataccg cgagacccac gctcaccggc tccagattta tcagcaataa accagccagc |
| 3601 | cggaagggcc gagcgcagaa gtggtcctgc aactttatcc gcctccatcc agtctattaa |
| 3661 | ttgttgccgg gaagctagag taagtagttc gccagttaat agtttgcgca acgttgttgc |
| 3721 | cattgctaca ggcatcgtgg tgtcacgctc gtcgtttggt atggcttcat tcagctccgg |
| 3781 | ttcccaacga tcaaggcgag ttacatgatc ccccatgttg tgcaaaaaag cggttagctc |
| 3841 | cttcggtcct ccgatcgttg tcagaagtaa gttggccgca gtgttatcac tcatggttat |
| 3901 | ggcagcactg cataattctc ttactgtcat gccatccgta agatgctttt ctgtgactgg |
| 3961 | tgagtactca accaagtcat tctgagaata gtgtatgcgg cgaccgagtt gctcttgccc |
| 4021 | ggcgtcaata cgggataata ccgcgccaca tagcagaact ttaaaagtgc tcatcattgg |
| 4081 | aaaacgttct tcggggcgaa aactctcaag gatcttaccg ctgttgagat ccagttcgat |
| 4141 | gtaacccact cgtgcaccca actgatcttc agcatctttt actttcacca gcgtttctgg |
| 4201 | gtgagcaaaa acaggaaggc aaaatgccgc aaaaaaggga ataagggcga cacggaaatg |
| 4261 | ttgaatactc atactcttcc tttttcaata ttattgaagc atttatcagg gttattgtct |
| 4321 | catgagcgga tacatatttg aatgtattta gaaaaataaa caaatagggg ttccgcgcac |
| 4381 | atttccccga aaagtgccac ctgacgtc |

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

All methods described herein can be performed in any suitable order unless otherwise indicated herein or otherwise clearly contradicted by context. The use of any and all examples provided herein, is intended merely to better illuminate the invention and does not pose a limitation on the scope of the invention unless otherwise claimed. No language in the specification should be construed as indicating any non-claimed element as essential to the practice of the invention.

Citations to a number of patent and non-patent references are made herein. The cited references are incorporated by reference herein in their entireties. In the event that there is an inconsistency between a definition of a term in the specification as compared to a definition of the term in a cited reference, the term should be interpreted based on the definition in the specification.

We claim:

1. An engineered bacterial strain comprising one or more heterologous hydrolase capable of degradation of at least a portion of a polyester material.

2. The engineered bacterial strain of claim 1, wherein the polyester material comprises polyethylene terephthalate (PET).

3. The engineered bacterial strain of claim 1, wherein the engineered bacterial strain is an engineered strain selected from Pseudomonas putida, Cupriavidus necator, Corynebacterium glutamicum, Zymomonas mobilis, Rhodococcus jostii, and/or Bacillus licheniformis.

4. The engineered bacterial strain of claim 3, wherein the engineered bacterial strain is derived from Pseudomonas putida KT2440.

5. The engineered bacterial strain of claim 1, wherein the one or more heterologous hydrolase is from Comamonas testosteroni.

6. The engineered bacterial strain of claim 1, wherein the one or more heterologous hydrolase is CtesDRAFT_PD1902 enzyme and/or CtesDRAFT_PD3135 enzyme.

7. The engineered bacterial strain of claim 1, wherein the one or more heterologous hydrolase comprises an amino acid sequence that is at least 80%, at least 85%, at least 90%, at least 95%, at least 98%, or at least 99% identical to the amino acid sequence of SEQ ID NO: 1.

8. The engineered bacterial strain of claim 1, wherein the engineered bacterial strain is engineered to express the heterologous hydrolase.

9. A method of fragmentation and/or degradation of at least a portion of a polyester material, comprising exposing the polyester material to one or more hydrolase from Comamonas testosteroni.

10. The method of claim 9, wherein the polyester material comprises PET.

11. The method of claim 9, wherein the one or more hydrolase is present in an engineered bacterial strain comprising the one or more heterologous hydrolase.

12. The method of claim 9, wherein the one or more heterologous hydrolase is capable of degradation of at least a portion of a polyester material.
